(19) United States
(12) Patent Application Publication
(10) Pub. No.: US 2024/0132438 A1
(43) Pub. Date: Apr. 25, 2024

(54) BIO-BASED POLYMERS FOR THE PURIFICATION OF HIGH COMMERCIAL VALUE CHEMICALS EXTRACTED FROM PLANTS, FOOD WASTE, AND NON-FOOD BIOMASS

(71) Applicants: Yagya Gupta, Newark, DE (US); Laura E. Beckett, Ingolstadt, DE (US); Sunitha Sadula, Wilmington, DE (US); Dionisios G. Vlachos, Newark, DE (US); LaShanda T. Korley, Middletown, DE (US)

(72) Inventors: Yagya Gupta, Newark, DE (US); Laura E. Beckett, Ingolstadt, DE (US); Sunitha Sadula, Wilmington, DE (US); Dionisios G. Vlachos, Newark, DE (US); LaShanda T. Korley, Middletown, DE (US)

(73) Assignee: University of Delaware, Newark, DE (US)

(21) Appl. No.: 18/490,069

(22) Filed: Oct. 18, 2023

Related U.S. Application Data
(60) Provisional application No. 63/417,369, filed on Oct. 19, 2022.

(51) Int. Cl. C07C 67/58; C08J 11/08

(52) U.S. Cl. CPC C07C 67/58; C08J 11/08; C08J 2333/26; C08J 2339/08

(57) ABSTRACT

Disclosed herein is a process for separating phenolic acids, comprising a step a) of contacting a feed containing at least two different phenolic acids (PA) with an extraction solvent to extract the at least two different PAs in a first PA containing liquid. The process also comprises a step b) of contacting the first PA containing liquid with a solid molecular imprinted polymer (MIP), such that the MIP captures a target PA from the at least two different PAs, to thereby form a first PA bound MIP dispersed in a second PA containing liquid, where the second PA containing liquid comprises at least one PA and none or a substantially lesser amount of the target PA originally present in the first PA containing liquid. The process further comprises a step c) of separating the first phenolic acid bound MIP from the second PA containing liquid, and a step d) of separating the target phenolic acid from the first PA bound MIP to obtain a recovered MIP, wherein the recovered MIP is substantially free of the target phenolic acid.

### CROSS-REFERENCE TO RELATED APPLICATION

This application claims priority to U.S. Provisional Patent Application No. 63/417,369, filed Oct. 19, 2022, the disclosure of which is being incorporated herein by reference in its entirety for all purposes.

### STATEMENT REGARDING FEDERALLY SPONSORED RESEARCH

This invention was made with government support under Grant No. NSF GCR CMMI 1934887 awarded by the National Science Foundation. The government has certain rights in the invention.

### BACKGROUND OF THE INVENTION

Food waste (FW) is a global challenge with approximately 17% of global food production, estimated to be 931 million tons annually, ending as waste (Forbes et al., Food Waste Index Report, 2021). Current FW management, such as anaerobic digestion, composting, fermentation, and animal feed, produces low-value products, i.e., biogas, compost, and ethanol. Alternative valorization strategies are required to reduce greenhouse gas emissions and the long processing times of current technologies.

The extraction of phenolic acids as natural antioxidants from FW is a lucrative alternative. Synthetic phenolic antioxidants, such as butylated hydroxyanisole, butylated hydroxytoluene, propyl gallate, and tert-butyl hydroquinone, and their transformation products have been shown to be toxic in animal studies and may threaten human health (Wang et al., Environ Res. 201 (2021) 111531; Liu et al., Environ Sci Technol. 54 (2020) 11706-11719). Hence, natural antioxidants, such as chlorogenic, caffeic, p-coumaric, and ferulic acids, are being increasingly used in the pharmaceutical, food, and cosmetic industries. These phenolic acids can also be converted to BTX (benzene, toluene, and xylene) which are currently produced from petroleum, but these are lower-value products.

Various mixtures of antioxidant powders extracted from plants and biomass are currently industrially produced. Various companies such as Nutragreen, Euromed, Cymbio Pharma, Flavor Trove, and Applied Food Sciences, produce coffee bean extracts containing caffeine, chlorogenic, and other hydroxycinnamic acids (Table 14). Kao Corporation (U.S. Pat. No. 8,309,150) reported a method to extract and purify 90% chlorogenic acid from raw or roasted coffee beans (FIG. 17). Kao's product is a mixture of nine mono-caffeoylquinic, feruloylquinic, and dicaffeoylquinic acids. The antioxidant capacity of mixtures depends on the components' molecular structures, intermolecular interactions, and concentrations. For instance, a mixture of acids extracted from green coffee beans has a lower antioxidant capacity than pure chlorogenic acid (Ramalakshmi et al., Food Research International. 41 (2008) 96-103). Thus, purifying target natural phenolic acids as replacements for synthetic antioxidants is desirable.

The purification of phenolic acids post-extraction is challenging because of their structural similarity, high boiling points close to each other, and similar chemical functionality. As a result, traditional separation methods, such as distillation, membrane separation, and ionic resin adsorption, are ineffective.

Molecular imprinted polymers (MIPs) achieve separations based on structural recognition. The target molecule (template) is imprinted on a constructed polymer and then washed out with a solvent to leave behind a cavity in the MIP (FIG. 2A). Due to its molecular structure and interactions with the target molecule, such as hydrogen bonding, van der Waals interactions, and electrostatic ionic interactions, the cavity selectively adsorbs the target molecule from a mixture. The MIP is then washed with a solvent to extract the target compound and regenerate the empty cavity. The solvent can then be evaporated to obtain the target compound in purified form. The high selectivity of the MIPs enable their diverse applications in chromatography, purification, sensing, detection, catalysis, and drug delivery. Previously, MIPs were employed for chlorogenic acid purification. Li et al. (Food Chem. 139 (2013) 1161-1167) developed an imprinted polymer monolithic stationary phase for purifying chlorogenic acid extracted from E. ulmodies leaves by selectively adsorbing the other compounds present in the extract. The approach provides low-purity chlorogenic acid, as the adsorbent does not preferentially adsorb all co-existing compounds. Hao et al. (Food Chem. 200 (2016) 215-222) and Zhao et al. (Talanta. 181 (2018) 271-277) conducted column chromatography with imprinted polymers to extract chlorogenic acid from spiked fruit juices and honeysuckle samples. These chromatographic methods are limited to a narrow range of operations and present scale-up challenges, rendering them useful only for analytical purposes. The implementation of a bio-based MIP for separating target phenolic acids post-extraction from FW has not been reported but has advantages over conventional processes, thus creating a need for a bio-based MIP.

### SUMMARY OF THE INVENTION

As noted above, FW is a profound challenge as 17% of global food production (i.e., 931 million tons) ends up as waste yearly. A cost-effective strategy is the extraction of high-value phenolic acids from FW, but their downstream purification is challenging owing to their similar chemical nature, high boiling points, and low concentrations in complex mixtures. Disclosed herein is a process for separating target phenolics post-extraction from FW using MIPs. It has been found that the stability of the monomer-template complex during the synthesis of the MIP is critical in determining the MIP selectivity and performance. COnductor like Screening MOdel for Real Solvents (COSMO-RS) and Hansen Solubility Parameters in Practice (HSPiP) computations were used to screen the interaction of 28 monomers and 13 porogenic solvents with chlorogenic acid as the target molecule, with experimental results revealing that itaconic acid (ITA), (the functional monomer), and tetrahydrofuran (THF) (the synthesis solvent), provide the highest reported observed separation factor. The resulting MIP exhibits superior selectivity towards chlorogenic acid in concentrated solutions of up to 1 mg/mL, and its high sensitivity to various functionalities enables the effective separation of all target phenolic acids. The MIP's performance was evaluated in different extraction solvents, and Fourier transform infrared (FTIR) studies revealed a polymer-solvent interaction to be a critical factor influencing its performance. Use of the MIP in obtaining chlorogenic acid in up to 92% purity from coffee beans and potato peel waste extractives is demonstrated herein below, and its reusability is evaluated. In contrast to the current industrial purification methodology that produces mixtures, the disclosed inventive process provides at least 11× higher economic value and 95% less carbon emissions based on lab-scale techno-economic analysis and carbon footprint calculations.

In an aspect of the invention, a process for separating phenolic acids, comprises a step a) of contacting a feed containing at least two different phenolic acids (PA) with an extraction solvent to extract the at least two different phenolic acids in a first PA containing liquid. The process also comprises a step b) of contacting the first PA containing liquid with a solid MIP, such that the MIP captures a target phenolic acid from the at least two different phenolic acids, to thereby form a first phenolic acid bound MIP dispersed in a second PA containing liquid, where the second PA containing liquid comprises at least one phenolic acid and none or a substantially lesser amount of the target phenolic acid originally present in the first PA containing liquid. The process further comprises a step c) of separating the first phenolic acid bound MIP from the second PA containing liquid, and a step d) of separating the target phenolic acid from the first phenolic acid bound MIP to obtain a recovered MIP, wherein the recovered MIP is substantially free of the target phenolic acid. In some embodiments, the process comprises repeating the steps b) to d) to extract remaining phenolic acid(s) of the at least two phenolic acids.

In an embodiment, the at least two phenolic acids differ from each other in at least one of functional group, aromatic ring substitution, polarity, and hydrogen bonding. In one embodiment, the target phenolic acid is chlorogenic acid. In another embodiment, the at least two different phenolic acids further comprise one or more of caffeic acid, p-coumaric acid, and ferulic acid.

The MIP has a BET surface area in the range of 80 to 250 m²/g and a BET pore size in the range of 5 to 11 nm. In some embodiments, the MIP in the second and subsequent sequence of steps b) to d) is the recovered MIP.

In an embodiment, the target phenolic acid has a separation factor of at least 1, wherein the separation factor is calculated as follows:

$$Seperation factor = \frac{Adsorption capacity of target phenolic acid}{\begin{matrix}Sum of adsorption capacity of all other phenolic \\ acids of the at least two phenolic acids\end{matrix}} ;$$

In various embodiments, the feed comprises one or more of raw, roasted, or spent coffee beans; potato peels; grapes; honeysuckle; apple; tomato; eggplant; carrot; and leaves from artichoke, E. ulmodies, tea, and tobacco. In some embodiments, the target phenolic acid is extracted at a purity of at least 80%.

In an embodiment of the process, the step of separating the target phenolic acid from the phenolic acid bound MIP comprises using ultrasonic assisted desorption in methanol, ethanol, 2-propanol, and/or tetrahydrofuran.

In another embodiment, the process further comprises preparing the MIP. The process comprises providing a polymerizable mixture comprising a pre-polymerization complex of at least one functional monomer and a target phenolic acid in at least one porogenic solvent. The process also comprises polymerizing the polymerizable mixture in the presence of a cross-linker, and the at least one porogenic solvent to generate the MIP, such that the target phenolic acid is non-covalently bound to the MIP; and washing the MIP with an extraction solvent to remove the target phenolic acid and to thereby form a MIP comprising molecular sized cavities adapted to selectively capture and bind the target phenolic acid.

In an embodiment, the step of providing a polymerizable mixture comprises selecting the at least one functional monomer and the at least one porogenic solvent based on their respective molecular interactions with the target phenolic acid. In some embodiment, the target phenolic acid has a solubility in the at least one porogenic solvent in a mole fraction range of 0.001 to 0.99, based on the total moles of the target phenolic acid and the porogenic solvent. In other embodiments, the porogenic solvent has a dielectric constant in a range of 5 to 50.

In some other embodiment, the at least one functional monomer has a solubility in the porogenic solvent in a mole fraction range of 0.01 to 0.99. The at least one functional monomer has the following Hansen Solubility parameters:
(i) a dispersion δD in the range of 15 to 21;
(ii) a polarity δP in the range of 5 to 15;
(iii) a hydrogen bond character δH in the range of 7 to 21; and
(iv) a Hansen Solubility Parameters in Practice distance from the target phenolic acid in the range of 0 to 10.

In an embodiment, the at least one functional monomer is selected from the group consisting of acrylamide, 4-vinyl pyridine, 2,6-diaminopyridine, itaconic acid, o-phenylenediamine, o-aminophenol, 2-hydroxyethyl methacrylate, p-aminostyrene, o-phthalic dialdehyde, acrylic acid, methacrylamide, N,N′-methylene bisacrylamide, methacrylic acid, N,N-dimethylacrylamide, allyl mercaptan, p-divinylbenzene, acrolein, 2-vinyl pyridine, N-vinyl-2-pyrrolidinone, acrylonitrile, methyl methacrylate, styrene, N,N-dimethylaminoethyl methacrylate, 4-ethyl styrene, (diethylamino)ethyl methacrylate, m-divinylbenzene, 3-aminopropyltriethoxysilane, tartaric acid, lactic acid, and combinations thereof.

In another embodiment, the at least one porogenic solvent comprises hexane, benzene, toluene, chloroform, tetrahydrofuran, dichloroethane, dichloromethane, 2-methoxyethanol, ethanol, methanol, N,N-dimethylformamide, acetonitrile, dimethyl sulfoxide (DMSO), or mixtures thereof.

In an embodiment, the at least one porogenic solvent comprises tetrahydrofuran, and the at least one functional monomer comprises acrylamide, o-aminophenol, itaconic acid, o-phenylenediamine, 2-hydroxyethyl methacrylate, or combinations thereof. In another embodiment, the target phenolic acid comprises chlorogenic acid, caffeic acid, p-coumaric acid, or ferulic acid; the functional monomer comprises itaconic acid; the radical initiator comprises 2,2-azobisisobutyronitrile, and the crosslinker comprises ethylene glycol dimethacrylate or 1,3-diisopropylbenzene.

In some embodiment of the process, the target phenolic acid and the functional monomer are present at a ratio in the range of 1:2 to 1:6. In other embodiments, the functional monomer and the crosslinker are present at a ratio in the range of 1:1 to 1:5.

### BRIEF DESCRIPTION OF THE DRAWINGS

FIG. 1 shows a process flow diagram for selective separation of target extractives from a mixture of phenolic acids using a molecular imprinted polymer, according to various embodiments of the present invention. PA in the figure stands for phenolic acid.

FIG. 2A (Prior Art) shows a schematic illustration of the preparation of a molecular imprinted polymer (MIP) synthesis.

FIG. 2B shows chemical structures of exemplary target molecules, functional monomers, and crosslinkers for use in the MIP synthesis shown in FIG. 2A, according to various embodiment of the present invention.

FIGS. 3A-3C show model-guided solvent and monomer selection for MIP synthesis. FIG. 3A shows dielectric constant of various solvents and their HSPiP predicted distance from chlorogenic acid. The dashed line is drawn for visual guidance. FIG. 3B shows HSPiP-predicted distance of various monomers from chlorogenic acid. FIG. 3C shows COSMO-RS computed surface charge density of different monomers at σ(e/Å²)=0 and 0.01. 2,6-DAP, o-AP, o-PHY, ITA, HEM, ACY, and 4-VP refer to 2,6-diaminopyridine, o-aminophenol, o-phenylenediamine, itaconic acid, 2-hydroxyethyl methacrylate, acrylamide, and 4-vinyl pyridine, respectively.

FIG. 4A shows single component adsorption capacity of MIPs made of different functional monomers with chlorogenic acid (template) and ethylene glycol dimethacrylate (EDGMA) (crosslinker) for 1 mg/mL chlorogenic acid in N,N-dimethylformamide (DMF) at 298 K for 2 h.

FIGS. 4B-4C show multicomponent adsorption capacity of MIPs made of different; monomer (FIG. 4B); and template (FIG. 4C) from a mixture containing an equal concentration of chlorogenic, caffeic, p-coumaric, and ferulic acids at 0.1 mg/mL in DMF at 298 K for 2 h. The MIPs P1, P2, P3, and P4 are described in Table 7. 2,6-DAP, o-AP, o-PHY, ITA, HEM, ACY, and 4-VP refer to 2,6-diaminopyridine, o-aminophenol, o-phenylenediamine, itaconic acid, 2-hydroxyethyl methacrylate, acrylamide, and 4-vinyl pyridine, respectively. CLA, CA, PCA, and FA are chlorogenic acid, caffeic acid, p-coumaric acid, and ferulic acid, respectively.

FIG. 5 shows scanning electron micrographs of MIPs synthesized with monomers: a) o-aminophenol, b) 2,6-diaminopyridine, c) 2-hydroxyethyl methacrylate, and d) o-phenylenediamine.

FIG. 6A shows FTIR spectrum of chlorogenic acid imprinted polymer with functional monomer itaconic acid and THF as the solvent at 1:3 monomer to crosslinker ratio.

FIGS. 6B-6C show scanning electron micrographs of imprinted polymer (FIG. 6B); and non-imprinted polymer (FIG. 6C) with itaconic acid as the functional monomer.

FIG. 7A shows time-dependent single component adsorption capacity of P2 for 1 mg/ml chlorogenic acid in DMF at 298 K; FIG. 7B shows experimental P2 adsorption isotherms (data points) and corresponding fitted Langmuir isotherm (solid line). The solid line has been added as a visual guide in FIG. 7A.

FIG. 8A shows separation factor of imprinted polymer P2 vs. concentration of a solution containing an equal weight percent mixture of phenolic acids (chlorogenic, caffeic, p-coumaric, and ferulic acid) in DMF at 298 K for 2 h. The line is a visual guide only, and the data labels represent chlorogenic acid adsorption capacity (mg/g).

FIG. 8B shows P2 adsorption capacity in equal concentration (0.1 mg/mL) mixtures of different target compounds in DMF at 298 K, and 2 h. M1 is a mixture containing an equal concentration of chlorogenic, caffeic, p-coumaric, and ferulic acids at 0.2 mg/mL. M2 is a mixture containing an equal concentration of caffeic, p-coumaric, and ferulic acids at 0.2 mg/mL. M3 is a mixture containing an equal concentration mixture of caffeic and p-coumaric acid at 0.2 mg/mL.

FIGS. 9A-9B show time-dependent single component adsorption capacity of P2 for 1 mg/ml chlorogenic acid at 298 K in water (FIG. 9A) and ethanol (FIG. 9B). The solid line has been added as a visual guide in FIGS. 9A and 9B.

FIG. 10A shows multicomponent adsorption capacity of imprinted polymer P2 for an equal concentration (0.1 mg/mL) mixture of acids (chlorogenic, caffeic, p-coumaric and ferulic acids) in ethanol and water at 298 K for 2 h and 30 min, respectively.

FIG. 10B shows FTIR absorbance of saturated mixture of DMF and polymer P2.

FIG. 11 shows FTIR spectrum of fresh and used polymer P2.

FIG. 12 shows time-dependent single component adsorption capacity of DMF on the polymer.

FIG. 13A shows adsorption capacity of imprinted and non-imprinted polymers. Single component adsorption capacity of imprinted polymer P2 and non-imprinted polymer (monomer: itaconic acid) for 1 mg/ml chlorogenic acid in DMF at 298K for 2 h.

FIG. 13B shows multicomponent adsorption capacity of imprinted polymer P2 and non-imprinted polymer (monomer: itaconic acid) for an equal concentration mixture of acids (chlorogenic, caffeic, p-coumaric, and ferulic acids) at 0.1 mg/ml in DMF at 298 K for 2 h.

FIG. 14 shows multicomponent adsorption capacity of the fresh imprinted polymer P2 and spent polymer P2 recycled up to three times from a mixture containing an equal concentration of chlorogenic, caffeic, p-coumaric, and ferulic acids at 0.1 mg/mL in DMF at 298 K for 2 h. Q is the adsorption capacity in mg/g.

FIGS. 15A-15B show P2 performance in multicomponent mixtures extracted at 298 K for 2 h from coffee waste (FIG. 15A) and potato peel waste (FIG. 15B).

FIG. 16 shows a process flow diagram for chlorogenic acid purification, extracted from coffee beans waste, using P2 according to embodiments of the present invention.

FIG. 17 (Prior Art) shows an industrial technology for producing 90% pure chlorogenic acids by Kao Corporation.

### DETAILED DESCRIPTION OF THE INVENTION

As used herein, the term “porogenic solvent” refers to a solvent used to dissolve all of the reagents that are used for polymer synthesis, including functional monomer(s), crosslinker(s), initiator(s), and molecular template(s), to then form a polymer matrix. Additionally, the porogenic solvent should help stabilize the pre-polymerization complex by promoting interactions between the functional monomer(s) and the molecular template(s).

As used herein, the term “phenolic acid containing liquid” is used interchangeably with “PA liquid”. For example, the term “first phenolic acid containing liquid” is used interchangeably with “first PA liquid,” “second phenolic acid containing liquid” is used interchangeably with “second PA liquid,” and “third phenolic acid containing liquid” is used interchangeably with “third PA liquid,” and so on.

As used herein, the term “bio-based molecular imprinted polymer” refers to a polymer/MIP derived from at least one bio-based monomer, which is a biomass-derived compound.

As used herein, the term “biomass-derived” is used interchangeably with “biologically-derived”, “bio-derived,” or “bio-based” and refers to compounds that are obtained from renewable resources, such as plants, and contain only or substantially renewable carbon, and none or a very minimal amount of fossil fuel-based or petroleum-based carbon.

As used herein, the term “molecularly imprinted polymer” or “MIP” is used interchangeably with “imprinted polymer,” “polymer,” “adsorbent polymer,” and “adsorbent.”

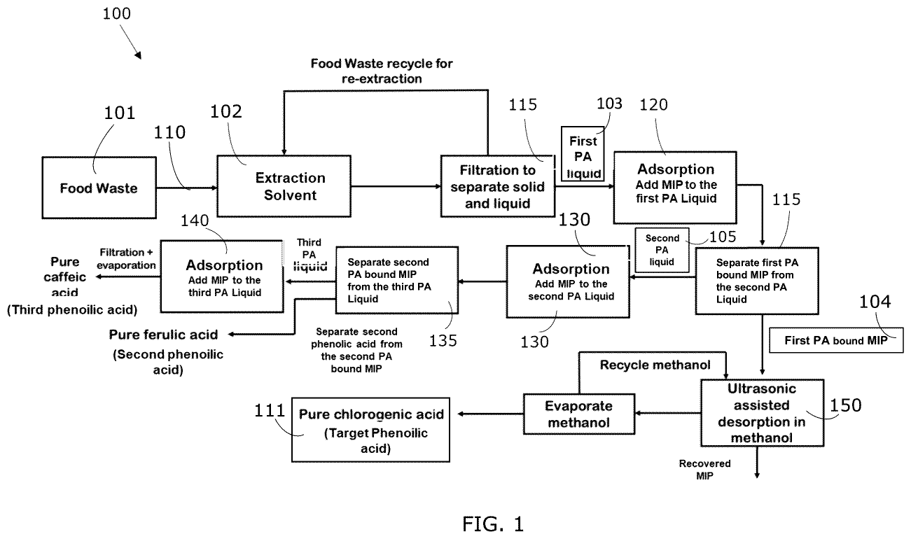

In an aspect of the present invention, disclosed herein is a process for separating a mixture of phenolic acids from food waste (FW). In an embodiment, a process 100 for separating phenolic acids is shown in FIG. 1. The process 100 comprises a step a) 110 of contacting a feed 101, containing at least two different phenolic acids (PA), with an extraction solvent 102 to extract the at least two different phenolic acids in a first PA containing liquid. In an embodiment, the feed 101 comprises FW. The step of contacting the feed 101 with extraction solvent 102 can be carried out at any suitable temperature for any suitable amount of time with stirring. In an embodiment, the step of contacting is carried out at a temperature in the range of 55 to 65° C. for 1.5 to 2.5 h. Generally, shorter times and lower temperatures are preferred for the extraction of at least two phenolic acids from the feed 101. In an embodiment, the step a) further comprises a filtration step 115 to separate the solid feed and the first PA containing liquid 103. In some embodiment, the filtration step 115 may be accompanied with reusing/recycling the solid spent feed from the filtration step 115 and contacting it with the extraction solvent for extraction of at least two different phenolic acids (PA) from the solid spent feed. The process 100 further comprises a step b) 120 of contacting the first PA containing liquid 103 with a solid molecular imprinted polymer (MIP), such that the MIP captures a target phenolic acid from the at least two different phenolic acids present in the first PA containing liquid, to thereby form a first PA bound MIP 104 dispersed in a second PA containing liquid 105. In an embodiment, the second PA containing liquid 105 comprises at least one phenolic acid and none or a substantially lesser amount of the target phenolic acid originally present in the first PA containing liquid 103. In some embodiments of the process 100, the filtration step 115 is optional and the step b) 120 of contacting the first PA containing liquid 103 with a solid MIP comprises contacting the first PA containing liquid 103 with a solid MIP in a mixture of the first PA containing liquid 103 and the solid spent feed, from which at least two phenolic acids have been extracted into the first PA liquid 103. The process 100 also comprises a step c) 125 of separating the first PA bound MIP 104 from the second PA containing liquid 105, and a step d) 150 of separating the target phenolic acid 111 from the first PA bound MIP 104 to obtain a recovered MIP. In an embodiment, the recovered MIP is substantially free of the target phenolic acid 111. In some embodiments, the steps b) to d) are repeated as many times as needed to extract any remaining phenolic acid(s) of the at least two phenolic acids present in the original feed 101 and the first PA containing liquid 103.

In one embodiment, the process 100 further comprises repeating step b)-d) as shown in FIG. 1. The process comprises a step 130 of contacting the second PA containing liquid 105 with a solid MIP, such that the MIP captures a second target phenolic acid from the at least two different phenolic acids, to thereby form a second PA bound MIP dispersed in a third PA containing liquid. The process also comprises a step 135 of separating the second PA bound MIP from the third PA containing liquid and separating the second phenolic acid from the second PA bound MIP. The process 100 may further comprise step 140 of contacting the third PA containing liquid with a solid MIP and extracting a third phenolic acid.

In an embodiment, the at least two phenolic acids differ from each other in at least one of functional group, aromatic ring substitution, polarity, and hydrogen bonding. Suitable examples of phenolic acids include, but are not limited to, chlorogenic acid, caffeic acid, p-coumaric acid, and ferulic acid. In other embodiments, the at least two phenolic acids may also comprise one or more of 3-caffeoylquinic acid, 4-caffeoylquinic acid, 5-caffeoylquinic acid, 3,4-dicaffeoylquinic acid, 3,5-dicaffeoylquinic acid, 4,5-dicaffeoylquinic acid, 3-feruloylquinic acid, 4-feruloylquinic acid, 5-feruloylquinic acid, 3-feruloyl-4-caffeoylquinic acid, and their derivatives. In an embodiment, the target phenolic acid is chlorogenic acid. In another embodiment, the at least two different phenolic acids present in the first PA containing liquid, other than the target phenolic acid of chlorogenic acid, comprise one or more of caffeic acid, p-coumaric acid, and ferulic acid.

In an embodiment of the process, as disclosed hereinabove, the MIP in a second sequence of steps b) to d) is the recovered MIP.

In another embodiment, the target phenolic acid has a separation factor of at least 1, wherein the separation factor is calculated as follows:

$$Seperation factor = \frac{Adsorption capacity of target phenolic acid}{\begin{matrix}Sum of adsorption capacity of all other phenolic \\ acids of the at least two phenolic acids\end{matrix}} ;$$

In other embodiments, the target molecule has a separation factor of at least 1.5, or 2.0, or 2.5, or 3.0, or 3.5, or 4.0. In some embodiments, the at least two phenolic acids other than the target phenolic acid has a separation factor of at least 0.01, or 0.05, or 0.1, or 0.15, or 0.20, or 0.25, 0.3, or 0.35, or 0.40, or 0.45, or 0.50, and less than that of the target phenolic acid, i.e., less than 4.0, or 3.5, or 3.0, or 2.5, or 2.0, or 1.5, or 1.0.

In various embodiments, the MIP has a BET surface area in the range of 50 to 300 m²/g, or 75 to 275 m²/g, or 80 to 250 m²/g and a BET pore size in the range of 4 to 20 nm, or 4.5 to 15 nm, or 5 to 12 nm.

Any suitable feed may be used, including, but not limited to, the feed comprising one or more of raw, roasted, or spent coffee beans; potato peels; grapes; honeysuckle; apple; tomato; eggplant; carrot; and leaves from artichoke, E. ulmodies, tea, and tobacco.

In an embodiment of the process, the target phenolic acid is extracted at a purity of at least 80%, or 85%, or 90%, using nascent MIP that has not been used before, from FW. In an embodiment of the process, the target phenolic acid is extracted at a purity of at least 50%, 55%, 60%, 65%, 70%, 75%, 80%, or 85%, or 90%, or 95% using recovered MIP that has been used and recovered at least once, or twice, or thrice. In an embodiment, the chlorogenic acid as the target phenolic acid is extracted at a purity of at least 75% when using nascent MIP, at least 60% when using recovered MIP that has been used and recovered once; at least 55% with recovered MIP that has been used and recovered two times, and at least 50% with recovered MIP that has been used and recovered three times. In another embodiment, the target phenolic acid of chlorogenic acid, is extracted at a purity of at least 60%, or 65%, or 70%, or 75%, or 80%, or 85%, or 90% from coffee waste used as the feed and nascent MIP.

Any suitable separation techniques may be used to separate the target phenolic acid from the phenolic acid bound MIP. In an embodiment, the step of separating the target phenolic acid from the phenolic acid bound MIP comprises using ultrasonic assisted desorption in methanol, ethanol, 2-propanol, and tetrahydrofuran. In another embodiment, other separation techniques may be used, including, but not limited to, soxhlet extraction, and simple solvent washing until the template is no longer detected.

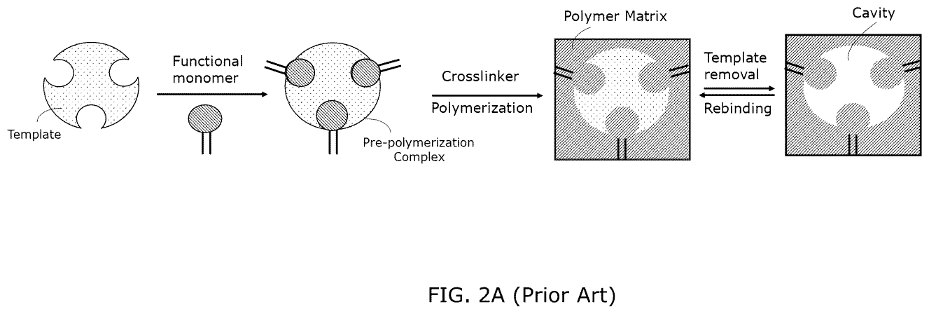

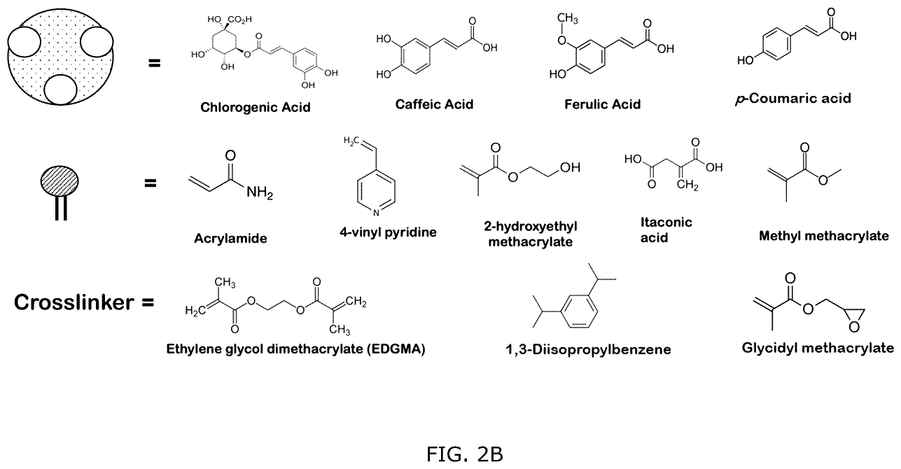

In an embodiment, the process further comprises preparing the MIP used in the process for separating phenolic acids. In an embodiment, a process for preparing the MIP is shown in FIG. 2A, which comprises providing a polymerizable mixture comprising a pre-polymerization complex of at least one functional monomer and a template of a target phenolic acid in at least one porogenic solvent. The process also comprises polymerizing the polymerizable mixture in the presence of a cross-linker, and the at least one porogenic solvent to generate the MIP, wherein the target phenolic acid is non-covalently bound to the MIP. The process further comprises washing the MIP with an extraction solvent to remove the target phenolic acid and to thereby form a MIP comprising molecular sized cavities adapted to selectively capture and bind the target phenolic acid. In an embodiment, the polymerization is carried out in an inert atmosphere such as nitrogen for any suitable amount of time in a range of 3 to 27 h, or 8 to 26 h, or 12 to 25 hr at a temperature in the range of 65 to 100° C., or 70 to 95° C., or 72 to 90° C.

In an embodiment, the step of providing a polymerizable mixture comprises selecting the at least one functional monomer and the at least one porogenic solvent based on their respective molecular interactions with the target phenolic acid. In another embodiment, the target phenolic acid has a solubility in the at least one porogenic solvent in a mole fraction range of 0.001 to 0.99, or 0.005 to 0.8, or 0.01 to 0.5, based on the total moles of the target phenolic acid and the porogenic solvent. In yet another embodiment, the functional monomer has a solubility in the at least one porogenic solvent in a mole fraction range of 0.01 to 0.99, or 0.03 to 0.7, or 0.04 to 0.5 at a temperature of about 298 K and 0.01 to 0.99, or 0.03 to 0.7, or 0.04 to 0.5 at about 333 K. In some embodiment, the at least one porogenic solvent has a dielectric constant in a range of 5 to 50, 6 to 30, or 7 to 15.

In another embodiment, the at least one functional monomer has the following Hansen Solubility parameters:
(i) a dispersion δD in the range of 15 to 21;
(ii) a polarity δP in the range of 5 to 15;
(iii) a hydrogen bond character δH in the range of 7 to 21; and
(iv) a Hansen Solubility Parameters in Practice (HSPiP) distance from the target phenolic acid in the range of 0 to 10.

In various embodiments, the at least one functional monomer comprises or is selected from the group consisting of acrylamide, 4-vinyl pyridine, 2,6-diaminopyridine, itaconic acid, o-phenylenediamine, o-aminophenol, 2-hydroxyethyl methacrylate, p-aminostyrene, o-phthalic dialdehyde, acrylic acid, methacrylamide, N,N′-methylene bisacrylamide, methacrylic acid, N,N-dimethylacrylamide, allyl mercaptan, p-divinylbenzene, acrolein, 2-vinyl pyridine, N-vinyl-2-pyrrolidinone, acrylonitrile, methyl methacrylate, styrene, N,N-dimethylaminoethyl methacrylate, 4-ethyl styrene, (diethylamino)ethyl methacrylate, m-divinylbenzene, 3-aminopropyltriethoxysilane, tartaric acid, lactic acid, and combinations thereof.

In one embodiment, the at least one porogenic solvent comprises hexane, benzene, toluene, chloroform, tetrahydrofuran, dichloroethane, dichloromethane, 2-methoxyethanol, ethanol, methanol, N,N-dimethylformamide, acetonitrile, dimethyl sulfoxide, or mixtures thereof.

In another embodiment, the at least one porogenic solvent comprises tetrahydrofuran, and the functional monomer comprises acrylamide, o-aminophenol, itaconic acid, o-phenylenediamine, 2-hydroxyethyl methacrylate, or combinations thereof.

In yet another embodiment, the target phenolic acid comprises chlorogenic acid, caffeic acid, p-coumaric acid, or ferulic acid; the functional monomer comprises itaconic acid; the radical initiator comprises 2,2-azobisisobutyronitrile; and the crosslinker comprises ethylene glycol dimethacrylate (EDGMA), and/or 1,3-diisopropylbenzene.

The target phenolic acid and the functional monomer may be present in any suitable amount. In an embodiment, the target phenolic acid and the functional monomer are present at a ratio in the range of 1:2 to 1:6, or 1:2.5 to 1:5, or 1.3 to 1:4.5. The functional monomer and the crosslinker may be present in any suitable amount, such as in a ratio in the range of 1:1 to 1:5, or 1:2 to 1:5, or 1:2.5 to 1:5, or 1:3 to 1.4.5.

An exemplary method of synthesizing a molecular imprinted polymer to selectively separate commercially valuable phenolic acids from a FW-extracted mixture is described below. The Hansen Solubility Parameters in Practice (HSPiP) model was used to identify monomers and solvents based on their molecular interaction with the target compounds for imprinted polymer synthesis. The COnductor like Screening MOdel for Real Solvents (COSMO-RS) multiscale simulation was used herein to generate sigma profiles to understand the molecular behavior of different monomers and their performance as a molecular imprinted polymer. Exemplary experiments that identify the best monomer and solvent for polymer synthesis, characterize the resulting polymers, and evaluate their performance in different phenolic mixtures, extraction solvents, and a broad concentration range are described below. Fourier transform infrared (FTIR) analysis confirms the structure of polymers and their interaction with the solvents. The separation strategy is demonstrated on potato peel and spent coffee bean waste. Lab-scale techno-economic analysis reveals the economic and environmental advantages of the proposed technology over commercial alternatives, distinguishing it from existing studies focused solely on analytical applications of imprinted polymers.

As used herein, when an amount, concentration, or other value or parameter is given as either a range, preferred range, or a list of upper preferable values and lower preferable values, this information is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

In some embodiments, the invention herein can be construed as excluding any element or process step that does not materially affect the basic and novel characteristics of the compositions or processes. Additionally, in some embodiments, the invention can be construed as excluding any element or process step not specified herein.

### EXAMPLES

Examples of the present invention will now be described. The technical scope of the present invention is not limited to the examples described below.

### Materials

Materials and their source are listed below:

Acrylamide (ACY) (GC grade (purity≥98%)), itaconic acid (ITA) (purity 99%), 2,6-diaminopyridine (2,6-DAP) (GC grade (purity≥97%), o-aminophenol (o-AP) (purity≥99%), ethylene glycol dimethacrylate (EDGMA) (purity≥98%), 2,2-azobisisobutyronitrile and caffeic acid (HPLC grade (purity≥98%) were purchased from Sigma Aldrich. p-Coumaric acid (purity≥98%), chlorogenic acid (purity≥99.45%), ferulic acid (purity 99.4%), 2-hydroxyethyl methacrylate (HEM) (purity≥97%), and o-phenylenediamine (o-PHY) (purity≥98%) were purchased from Fisher Scientific. ASTM-Type 1 grade deionized (DI) water (Milli-Q® Direct) was used in all experiments. Solvents N,N-dimethylformamide (DMF) (HPLC grade, purity≥99.9%), methanol (ACS reagent, purity≥99.5%), and ethanol (ACS reagent, purity≥99.5%) were obtained from Sigma Aldrich. Tetrahydrofuran (THF) (HPLC grade, purity≥99.5%) was obtained from Fisher Scientific.

### Methods

### Synthesis of Molecularly Imprinted Polymers and Non-Imprinted Polymers

The template molecule and a functional monomer at a ratio of 1:4 were added to 5 mL of THF. The mixture was stirred at room temperature in nitrogen for 30 min. 0.03 g of 2,2-azobisisobutyronitrile recrystallized with methanol and crosslinker EDGMA were added to the mixture such that the monomer and crosslinker weight by volume was 53.3%. The monomer-to-crosslinker ratio was 1:3. The solution was purged under nitrogen for 15 min and sealed. The polymerization proceeded for 24 h at 75° C. The resulting polymer was ultrasonicated in methanol for 1 h to remove the template molecule and dried under vacuum at 50° C. to thereby produce a molecularly imprinted polymer (MIP). The template extraction efficiency was determined to be 96% using high-performance liquid chromatography (HPLC). The polymer was grounded using a Thomas Wiley® Mini Cutting Mills to a powder of size <0.5 mm. A non-imprinted polymer was synthesized with the same methodology without using a template molecule and ultrasonication step.

### Adsorbent (MIP) Characterization

The porosity of the synthesized polymers was determined by nitrogen gas sorption at —196° C. using the Micromeritics ASAP 2020 Brunauer Emmett Teller (BET) Analyzer. TGA was performed using a TA instruments Q600 SDT thermogravimetric analyzer and differential scanning calorimeter (DSC) for a temperature program of 30 to 700° C. at a heating rate of 10 K min⁻¹ under air (30 mL min⁻¹). Scanning electron microscopy (SEM) was conducted using an AURIGA 60 Crossbeam FIB-SEM with an acceleration voltage of 3 kV. The specimens were coated with a thin (˜5 nm) conductive layer of Pd/Au to minimize sample charging using a vacuum sputter coater (Denton Desk IV, Denton Vacuum, LLC). A Specac Golden Gate diamond attenuated total reflectance (ATR) unit gave the FTIR spectra, with a 4 cm⁻¹ resolution in the 4000-1000 cm⁻¹ range using a Thermo Scientific Nicolet iS50 spectrometer instrument equipped with an MCT-B detector. Solutions (1 to 0.125 mg/μl) of chlorogenic acid and a saturated polymer mixture in a solvent (DMF, water, or ethanol) were analyzed.

The swelling ratio of the polymer was measured in DMF, water, and ethanol. 100 mg of dry polymer was weighed in a tube, and 1-1.5 mL of solvent was added. The tube was sealed, stirred for 2 h at 25° C., and centrifuged. The supernatant was drained, and the excess solvent was wiped off. The wet polymer was weighed, and the swelling ratio was estimated (Eq. 1).

$$Swelling ratio = \frac{Weight of wet polymer - weight of dry polymer}{Weight of dry polymer} \qquad ( 1 )$$

### Quantification of Phenolic Compounds

The standard acids concentration was quantified using HPLC using a Waters e2695 separations module coupled to a Waters 2414 refractive index meter and a Waters 2998 photodiode array detector. An Agilent Zorbax SB-C18, 250 mm column was used at 323 K, using solvent A (pure methanol) and solvent B (1% formic acid in water) as a mobile phase flowing at 0.8 mL/min. A gradient method was set up for 0% B to reach 95% B in 35 min. The concentration was calculated from the absorbance peak area measured between 320 to 380 nm at the respective retention time based on pure compound calibrations.

The FW-extracted acids were identified, and their concentration was quantified using Ultra performance liquid chromatography-mass spectrometry (UPLC-MS) on a Q-orbitrap mass spectrometer. A Waters Acquity UPLC BEH C18 column (1.7 μm^{2.1×30} mm) was used with solvent A (water containing 0.1% formic acid) and solvent B (acetonitrile containing 0.1% formic acid) as the mobile phase flowing at 0.5 mL/min. A gradient method was set up for 0% B to reach 95% B in 5 min.

### Single And Multi-Component Adsorption

For single component adsorption, a stock solution of chlorogenic acid in a solvent at 0.4, 5, 8, 10, 20, 50, and 100 mg/mL was made. For multicomponent adsorption, a stock solution of an equal concentration (in the range 0.1-30 mg/mL) mixture of chlorogenic, p-coumaric, caffeic, and ferulic acids was made in DMF, water, and ethanol. 2 mg of polymer was added to a vial containing 2 mL of stock solution. The mixture was stirred for 2 h at 298 K. All experiments were conducted in triplicates. 0.45 μm syringe filters were used for sampling. HPLC was used for quantification.

Extraction from FW

In Gupta et al. (Sep Purif Technol., 316 (2023)), DMF was determined to be an excellent solvent for extracting target phenolics owing to its superior polarity and hydrogen bond-accepting character. Thus, 20 mL of DMF and 1 g of FW feedstock (spent coffee bean or potato peel waste) were added to a round bottom flask. The target compounds are thermally degradable. Thus, the mixture was stirred and heated to 60° C. for 2 h (short times and lower temperatures). The solution was then filtered and quantified on ultra-performance liquid chromatography-mass spectrometry (UPLC-MS).

Adsorption from FW Extract

10 mg of adsorbent MIP was added to 10 mL of FW extracted solution. The mixture was stirred for 2 h at 298 K. Duplicate experiments were conducted. 0.45 μm syringe filters were used for sampling. The samples were quantified using UPLC-MS.

### Computations

The HSPiP (HSPiP 5.3.05) was used for the selection of monomers and solvents for polymer synthesis. The HSPiP distance (Ra) between two compounds provides their ‘likeness’: Ra²=4(δD₁-βD₂)²+(δP₁-βP₂)²+(δH₁-βH₂)² where δD, δP, and δH is dispersion, polar and hydrogen bonding energy. The uncertainty associated with the experimentally determined HSP and predicted distance are ±0.5 (MPa)^{0.5} and ±1 (MPa)^{0.5}, respectively (Lehnert et al, Applied Sciences (Switzerland). 10 (2020) 4266). Given its approximations, the ADF COSMO-RS implementation in the ADF2020.101 modeling suite was used to generate monomers' σ-profiles to understand their molecular behavior. The molecules were optimized in a vacuum using density functional theory (DFT) for sigma profile generation. For geometry optimization, the TZP small-core basis set, the Becke-Perdew (GGA: BP86) functional, the scalar ZORA, and the numerical integration quality of 4 with an energy convergence criteria 10⁻⁵ Ha were used. The approximations of COSMO-RS in the acid dissociation factor, long-range interactions, weak intermolecular forces, and molecule conformations affect its accuracy. Despite these limitations, it is believed that the computational tools can rapidly screen molecular databases to guide experimental efforts. Experiments are, however, necessary for validating and identifying the best system.

In the following sections, the computations-aided MIP design, its experimental assessment, and insights into the optimized MIP's morphological, molecular, and thermal properties are discussed. The selectivity of the MIP in a mixture of target acids and its concentration range of effective separation were analyzed, and then its versatility was determined as an adsorbent by evaluating its stability and performance in common extraction solvents. The MIP's suitability for large-scale applications was assessed by examining its reusability and efficacy in selectively purifying chlorogenic acid (model target acid) from two FW feedstocks. Finally, the economic and environmental advantages of the disclosed inventive method was evaluated and contrasted it with existing commercial technology.

### Example 1

### Polymer Design

Stable pre-polymerization complexes are crucial in non-covalent imprinting technology to obtain selective recognition sites. The monomer, template, and solvent interactions dictate the stability of such complexes.

### HSPiP and COSMO-RS Predictions

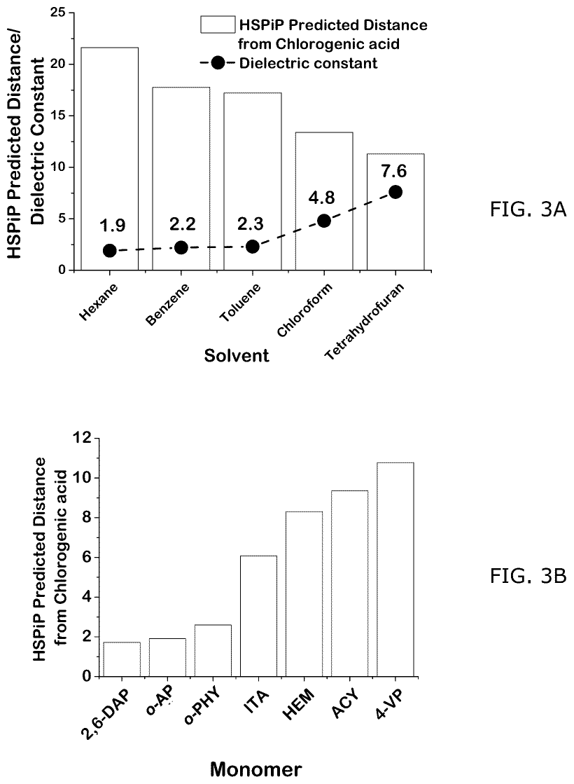

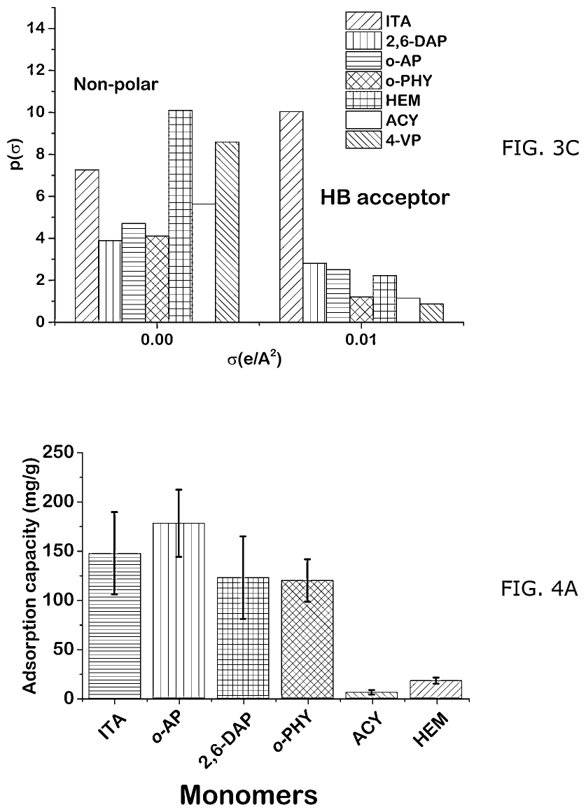

For solvent selection, dielectric constants compiled from the literature was used as a measure of their polarity. Since the template is polar (see Table 1 for target compounds' HSP), polar solvents would hinder its interactions with the monomer. In contrast, a non-polar solvent would enhance the stability of the pre-polymerization complex. Thus, 5 of 13 recommended porogenic solvents (see their dielectric constants and HSP in Table 2) with the lowest dielectric constants were selected (FIG. 3A). As the solvent brings monomer and template in one phase, their solubility in the solvent is also important. The HSPiP (FIG. 3A) and COSMO-RS (see Table 3 for solubility estimates) were used to estimate the likeability and solubility of chlorogenic acid (model template) and target monomers in the selected solvents. Non-polar solvents like hexane, benzene, toluene, and chloroform provide very low solubility for the monomers and chlorogenic acid, making polymer synthesis non-viable. THF provides good solubility and has a sufficiently low dielectric constant to stabilize the complex. Consequently, THF was selected for imprinted polymer synthesis.

TABLE 1
The Hansen Solubility Parameters of target compounds.

| Target Compound | δD | δP | δH |
|---|---|---|---|
| Chlorogenic acid | 19.83 | 9.47 | 16.75 |
| Caffeic acid | 20.69 | 8.26 | 19.04 |
| p-Coumaric acid | 20.07 | 7.59 | 16.55 |
| Ferulic acid | 19.8 | 7.56 | 15.76 |

TABLE 2
List of recommended porogenic solvents, their dielectric constants,
and Hansen Solubility Parameters. (Biosens Bioelectron. 107
(2018) 203-210; TrAC—Trends in Analytical Chemistry. 128
(2020) 115923; Chem Soc Rev. 45 (2016) 2137-2211).

| | | δD | | δH |
|---|---|---|---|---|
| | Dielectric | (Disper- | δP | (Hydrogen |
| Solvent | constant | sion) | (Polarity) | bonding) |
| Hexane | 1.9 | 14.9 | 0 | 0 |
| Benzene | 2.2 | 18.4 | 0 | 2 |
| Toluene | 2.3 | 18 | 1.4 | 2 |
| Chloroform | 4.8 | 17.8 | 3.1 | 5.7 |
| Tetrahydrofuran | 7.6 | 16.8 | 5.7 | 8 |
| (THF) | | | | |
| Dichloroethane | 8.93 | 16.5 | 7.8 | 3 |
| Dichloromethane | 9.1 | 17 | 7.3 | 7.1 |
| 2-Methoxyethanol | 16.94 | 16 | 8.2 | 15 |
| Ethanol | 22.4 | 15.8 | 8.8 | 19.4 |
| Methanol | 32.6 | 14.7 | 12.3 | 22.3 |
| N,N-dimethylformamide | 36.71 | 17.4 | 13.7 | 11.3 |
| (DMF) | | | | |
| Acetonitrile | 37.5 | 15.3 | 18 | 6.1 |
| Dimethyl sulfoxide | 46.68 | 18.4 | 16.4 | 10.2 |
| (DMSO) | | | | |

TABLE 3
COSMO-RS solubility in mole fraction of functional monomers
in the selected 5 solvents at about 298 K and about 333 K.

| Solubility (mole fraction) | | Hexane | Benzene | Toluene | Chloroform | THF |
|---|---|---|---|---|---|---|
| o-aminophenol | 298.15 K | 0.001 | 0.025 | 0.016 | 0.027 | 0.554 |
| | 333.15 K | 0.014 | 0.491 | 0.392 | 0.485 | 0.723 |
| 2,6-diaminopyridine | 298.15 K | 0 | 0 | 0 | 0 | 0.311 |
| | 333.15 K | 0 | 0.007 | 0.005 | 0.011 | 0.396 |
| itaconic acid | 298.15 K | 0 | 0 | 0 | 0 | 0.389 |
| | 333.15 K | 0 | 0.002 | 0.001 | 0.002 | 0.460 |
| o-phenylenediamine | 298.15 K | 0.002 | 0.028 | 0.018 | 0.041 | 0.412 |
| | 333.15 K | 0.018 | 0.429 | 0.318 | 0.446 | 0.625 |
| acrylamide | 298.15 K | 0 | 0 | 0 | 0 | 0.398 |
| | 333.15 K | 0 | 0.01 | 0.006 | 0.916 | 0.922 |
| chlorogenic acid | 298.15 K | 0 | 0 | 0 | 0 | 0.001 |
| | 333.15 K | 0 | 0 | 0 | 0 | 0.001 |

Acrylamide and 4-vinylpyridine are standard functional monomers for synthesizing polymers imprinted with polar bioactive compounds. For selection, a list of 28 previously used monomers was compiled for synthesizing imprinted polymers and HSPiP was applied to identify the ones interacting strongly with chlorogenic acid (see Table 4 for a comprehensive list and their HSP). The analysis showed acrylamide being superior to 4-vinyl pyridine and 7 monomers superior to acrylamide with amines being predominant (4 out of 7). Then, the top 5 monomers (FIG. 3B) were selected to provide insights into monomer selection and conduct experiments. Monomers of high polarity (δP) and HB character (δH) interact strongly with the template, given its polar and HB donor nature. HSPiP indicates that amines have the highest dispersion parameter (δD). However, itaconic acid has a higher δH, and acrylamide has a higher δP. The HSP weighs its dispersion component more compared to δP and δH. As a result, the trend is 2,6-diaminopyridine>o-aminophenol>o-phenylenediamine>itaconic acid>2-hydroxyethyl methacrylate>acrylamide>4-vinylpyridine. The σ-profiles (FIG. 3C) reveal that the amines, except 4-vinyl pyridine, have lower non-polar (at σ(e/Å²)=0) surface charge density (ρ(σ)) and overlapping profiles, indicating higher polarity and similar performance consistent with the HSPiP predictions. Itaconic acid has the highest HB acceptor charge density (at σ(e/Å²)>0.0079), connoting stronger interactions than amines in contrast to HSPiP. Finally, 2-hydroxyethyl methacrylate and 4-vinyl pyridine have low polarity and HB acceptor character and interact weakly than acrylamide. Consequently, itaconic acid is believed to exhibit superior performance than the amines.

TABLE 4
List of 28 monomers, their Hansen Solubility Parameters,
and HSPiP predicted distance from chlorogenic acid.

| | | | | HSPiP distance |
|---|---|---|---|---|
| | | | | from chlorogenic |
| Monomers | δD | δP | δH | acid |
| 2,6-diaminopyridine | 20.5 | 10.4 | 17.3 | 1.7213 |
| o-phenylenediamine | 20.5 | 7.7 | 15.4 | 2.5982 |
| o-aminophenol | 19.5 | 7.8 | 16.1 | 1.9097 |
| itaconic acid | 17.3 | 9.1 | 20.1 | 6.0797 |
| 2-hydroxyethyl methacrylate | 16.8 | 7.3 | 11.5 | 8.3063 |
| p-aminostyrene | 19 | 5.3 | 8.8 | 9.1294 |
| o-phthalic dialdehyde | 19.6 | 9.9 | 7.5 | 9.2714 |
| acrylamide | 15.8 | 12.1 | 12.8 | 9.3532 |
| acrylic acid | 16.6 | 6.4 | 10 | 9.8345 |
| methacrylamide | 15.8 | 11 | 11.6 | 9.6864 |
| N,N-methylene bisacrylamide | 18.7 | 17.8 | 11.5 | 10.1024 |
| methacrylic acid | 15.8 | 2.8 | 12 | 11.4897 |
| 4-vinyl pyridine | 18.1 | 7.2 | 6.8 | 10.7762 |
| N,N-dimethylacrylamide | 17.1 | 10.4 | 7.6 | 10.6957 |
| allyl mercaptan | 16.4 | 6.2 | 7.9 | 11.6651 |
| p-divinylbenzene | 18.6 | 1 | 7 | 13.1474 |
| acrolein | 15.5 | 10 | 8.6 | 11.9037 |
| 2-vinyl pyridine | 18.2 | 5.8 | 5.2 | 12.5498 |
| N-vinyl-2-pyrrolidinone | 16.4 | 9.3 | 5.9 | 12.8378 |
| acrylonitrile | 16 | 12.8 | 6.8 | 12.9910 |
| methyl methacrylate | 15.8 | 6.5 | 5.4 | 14.2340 |
| methylmethacrylate | 15.8 | 6.5 | 5.4 | 14.2340 |
| styrene | 18.6 | 1 | 4.1 | 15.4212 |
| N,N-dimethylaminoethyl | 16 | 3.8 | 5.2 | 14.9742 |
| methacrylate | | | | |
| 4-ethyl styrene | 17.7 | 2.7 | 3.3 | 15.6487 |
| (diethylamino)ethyl methacrylate | 15.8 | 3.6 | 4.4 | 15.8727 |
| m-divinylbenzene | 17.8 | 2.1 | 2.8 | 16.2911 |
| 3-aminopropyltriethoxysilane | 14.1 | 3.9 | 4 | 18.0255 |
| tartaric acid | 17.8 | 14 | 29.2 | 13.8566 |
| lactic acid | 17 | 8.3 | 28.4 | 13.0048 |

### Experimental Verification of Model Predictions

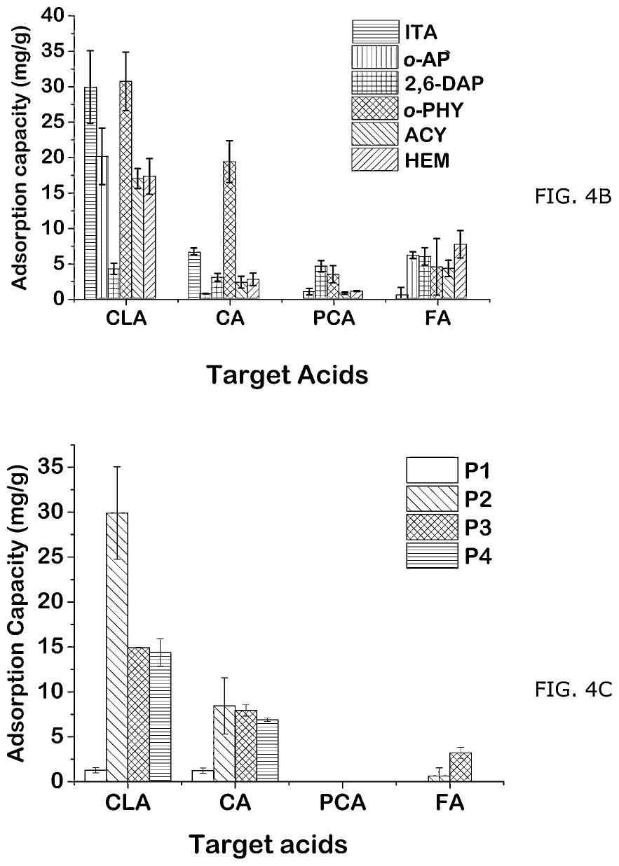

Experimentally, the MIPs synthesized with the selected 5 monomers provided higher single-component adsorption capacity than the polymer with acrylamide as the functional monomer (FIG. 4A). 2-hydroxyethyl methacrylate provides the lowest adsorption capacity (approximately 3 times that of acrylamide). The rest offered at least 20 times higher adsorption capacity than the literature standard. The adsorption capacity of the polymers made of the top four monomers was comparable, with minor variations in single-component adsorption, and are ranked as o-aminophenol>itaconic acid>2,6-diaminopyridine≈o-phenylenediamine.

In contrast to single component data, the multicomponent adsorption (FIG. 4B) reveals itaconic acid's superior performance, followed by o-aminophenol≈acrylamide>2-hydroxyethyl methacrylate>o-phenylediamine>2,6-diaminopyridine (see Table 5 for their separation factors). Only itaconic acid provides a higher separation factor (approximately 2 times) (Eq. 2) for the template (chlorogenic acid) than acrylamide. As discussed above, the synthesized amine polymers are porous (see their BET surface area in Table 6) but lack molecular selectivity, potentially due to lower HB character and over-estimation by HSPiP. Thus, itaconic acid was selected as a suitable monomer for efficient separation.

$$Seperation factor = \frac{Adsorption capacity of target molecule}{\begin{matrix}Sum of adsorption capacity of all other \\ compounds in the mixture\end{matrix}} \qquad ( 2 )$$

TABLE 5
Separation factors of target phenolic acid extractives using
polymers synthesized with different monomers, chlorogenic
acid (template), EDGMA (crosslinker), THF, and monomer to
crosslinker ratio as 1:3 from a mixture containing an equal
concentration of (chlorogenic, caffeic, p-coumaric and ferulic
acid at 0.1 mg/ml in DMF at 298 K for 2 h.

| | Separation factor of extractive | | | |
|---|---|---|---|---|
| | Chlorogenic | Caffeic | p-Coumaric | Ferulic |
| Monomer | acid | acid | acid | acid |
| itaconic acid (ITA) | 4.10 | 0.21 | 0 | 0.02 |
| o-phenylenediamine (o- | 1.12 | 0.50 | 0.06 | 0.08 |
| PHY) | | | | |
| 2,6-diaminopyridine | 0.31 | 0.20 | 0.34 | 0.50 |
| (2,6-DAP) | | | | |
| o-aminophenol (o-AP) | 2.6 | 0.02 | 0.04 | 0.28 |
| acrylamide (ACY) | 2.32 | 0.10 | 0.03 | 0.21 |
| 2-hydroxyethyl | 1.51 | 0.10 | 0.04 | 0.36 |
| methacrylate (HEM) | | | | |

TABLE 6
BET surface area and pore size.

MIP

| | BET Surface | BET Pore |
|---|---|---|
| Monomers | area (m²/g) | size (nm) |
| 2,6-diaminopyridine (2,6-DAP) | 219.6 | 7.83 |
| o-phenylenediamine (o-PHY) | 190.6 | 8.23 |
| o-aminophenol (o-AP) | 157.3 | 8.12 |
| itaconic acid (ITA) | 152.2 | 10.64 |
| 2-hydroxyethyl methacrylate | 86.6 | 9.15 |
| (HEM) | | |
| acrylamide (ACY) | 155.3 | 6.89 |
| Non-imprinted itaconic acid | 235.5 | 10.28 |

Next, all target compounds were evaluated as prospective templates. Four polymers (P1, P2, P3, and P4) were synthesized (see Table 7 for polymer description and their separation factors) with caffeic, chlorogenic, p-coumaric, and ferulic acid as the template, respectively (FIG. 4C). Ideally, imprinted polymers, MIP, should demonstrate the highest selectivity toward the corresponding template. However, experiments showed that all polymers prefer chlorogenic acid and reject p-coumaric acid regardless of the template. Without wishing to be bound by any particular theory, it is believed that the molecular interaction of the MIP (adsorbent) with the structurally analogous compounds (adsorbate) in a mixture leads to the observed performance. Specifically, HB is a characteristic parameter of the adsorbate-adsorbent interaction. The molecular structure of the target compounds (FIG. 2B) shows that chlorogenic acid has the highest number of HB sites, and p-coumaric acid has the lowest. Thus, chlorogenic acid interacts strongly with the adsorbent occupying the active sites in all polymers. Similarly, p-coumaric acid interacts weakly with the polymer, promoting the adsorption of other molecules. Consequently, the performance of P1, P3, and P4 is compromised, making P2 a suitable choice for the purification of target acids from a mixture.

TABLE 7
Separation factor of target phenolic acid extractives
for polymers made of different templates, itaconic acid
(monomer), EDGMA (crosslinker), THF, and monomer to crosslinker
ratio as 1:3 from a mixture containing an equal concentration
of (chlorogenic, caffeic, p-coumaric and ferulic acid
at 0.1 mg/ml in DMF at 298 K for 2 h.

| | | Separation | Separation | Separation | Separation |
|---|---|---|---|---|---|
| | | factor for | factor for | factor for | factor for |
| | | chlorogenic | caffeic | p-coumaric | ferulic |
| MIP | Template | acid | acid | acid | acid |
| P1 | Caffeic acid | 1.02 | 0.97 | 0 | 0 |
| P2 | Chlorogenic | 4.10 | 0.21 | 0 | 0.02 |
| | acid | | | | |
| P3 | p-Coumaric | 1.34 | 0.44 | 0 | 0.14 |
| | acid | | | | |
| P4 | Ferulic acid | 2.07 | 0.48 | 0 | 0 |

### Characterization of the Synthesized Polymers

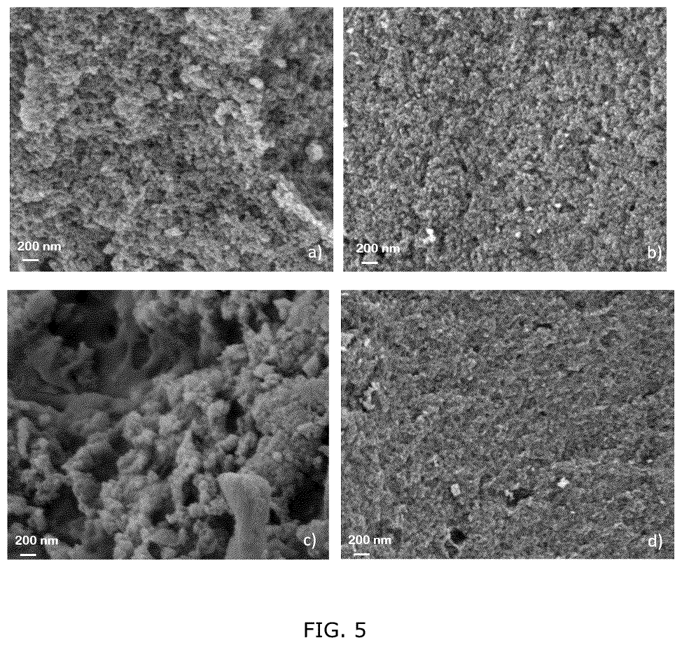

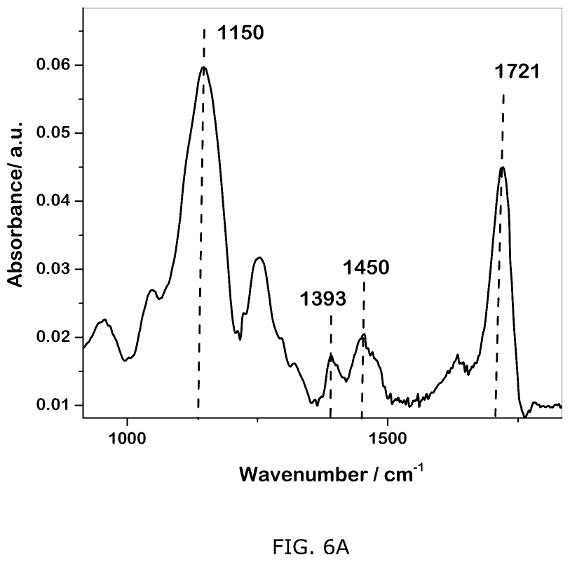

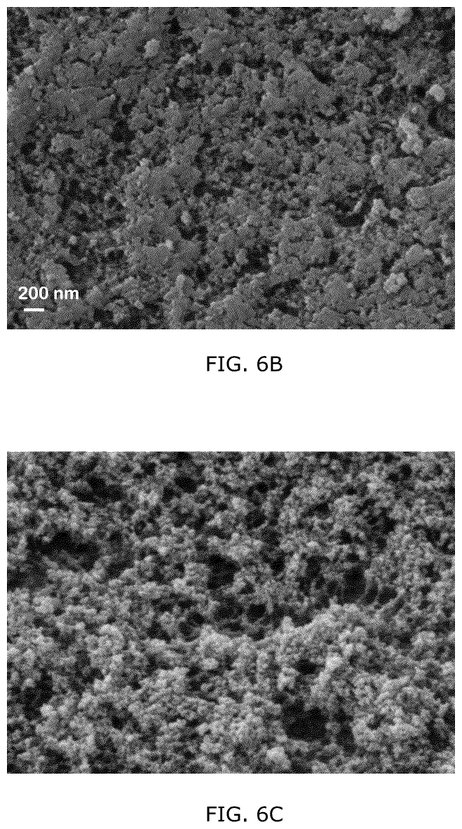

The morphology of the synthesized polymers was characterized using SEM (FIGS. 5, 6B and 6C) and the molecular structure was confirmed using FTIR. The IR spectrum (FIG. 6A) shows the itaconic acid characteristic peaks at 1721 cm⁻¹ (C=0 stretching band) and 1393 cm⁻¹ (OH bending) and crosslinker characteristic peak at 1150 cm⁻¹ (C—O—C stretching). The presence of the C-C stretching band at 1450 cm⁻¹ provides evidence for crosslinked structure and formation of the target polymer. SEM for imprinted (FIG. 6B) and non-imprinted polymer (FIG. 6C) (functional monomer:itaconic acid) depict porous materials confirming their suitability as adsorbents. The successful removal of the template chlorogenic acid was verified using HPLC analysis (see methods), as similar functional groups (—COON, —OH) limit the identification of chlorogenic acid in the polymer using IR. The polymer's swelling ratio is 0.09±0.02, 0.16±0.08, and 0.50±0.01 in water, ethanol, and DMF. The low swelling ratio of the polymer provides strong evidence for having a highly crosslinked structure. The BET surface area of the imprinted and non-imprinted polymer is 152.22 and 235.51 m²/g, with average pore sizes of 10.64 and 10.28 nm respectively. The polymers are mesoporous with a Type IV adsorption isotherm indicating the formation of a monolayer followed by multilayer adsorption (see below). The TGA curve for the polymer with itaconic acid (not shown) shows ˜2 wt % loss until 136° C., possibly due to volatilization of absorbed moisture or THF. The polymer gradually loses another ˜4 wt % between 136° C. and 300° C., corresponding to the removal of the remaining functional monomer (a boiling point of 268° C.) and crosslinker (boiling point of 235° C.). A steep loss at higher temperatures (300-500° C.) was observed, indicating polymer degradation. Hence, the synthesized polymer is thermally stable up to 300° C. under the present conditions.

### Determination of Equilibrium Time of Target Acids

A stock solution of 1 mg/ml chlorogenic acid in a solvent (DMF, water, or ethanol) was prepared. 2 mg of the synthesized polymer was added to a vial containing 2 ml of stock solution. The mixture was stirred at 298 K for 10, 30, 60 min, and up to 460 min with 1 h intervals in DMF, 10, 20, 30, 60 min, and up to 360 min with 1 h intervals in water, and 10, 40, 60, 120, and 180 min in ethanol. All experiments were run in triplicates. 0.45 μm syringe filters were used for sampling from each vial. The samples were analyzed using HPLC.

### Adsorption Isotherm and Time-Dependent Adsorption Capacity

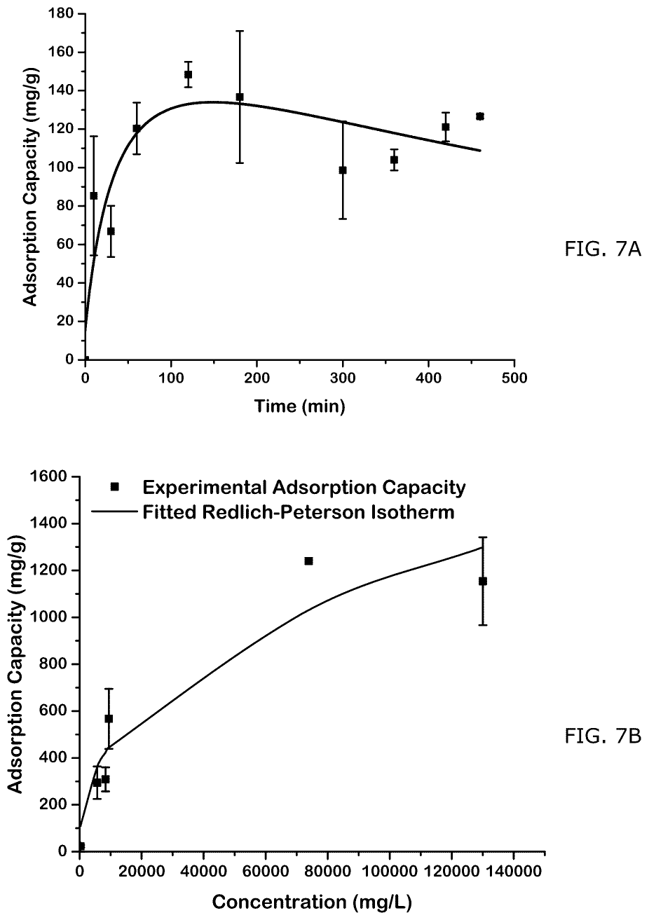

Time-dependent adsorption was estimated to determine system equilibrium (FIG. 7A). The adsorption capacity increases until 2 h and fluctuates at longer times by ˜15%. The interplay of extraction solvent, adsorbent, and adsorbate is essential to understand the stability and efficacy of the polymer (see below). The solvent-polymer interaction dictates adsorption and potentially causes the observed variation. It was deemed that 2 h is sufficient for P2 to attain equilibrium as it achieves maximum adsorption capacity with minimal variations thereafter.

The Redlich-Peterson model (Eq. 3) adequately describes the adsorption of chlorogenic acid on P2 (FIG. 7B), indicating multilayer and monolayer adsorption consistent with the BET isotherm. The model assumes that the adsorbent surface is not completely uniform, and the adsorption energy of the molecules varies across the surface. The exponent reflects the degree of heterogeneity of the surface, with a value of 0.6 indicating that the surface is relatively heterogeneous.

$${q}_{e} = \frac{{K}_{r} {C}_{e}}{1 + α {C}_{e}^{β}} \qquad ( 3 )$$

where qₑ is the adsorption capacity at concentration Cₑ, Kᵣ is the Redlich-Peterson isotherm constant in L/g, α is constant in L/mg, and β is an exponent ranging between 0 & 1.

### Range of Selective Adsorption and P2 Selectivity for Mixtures

The P2 batch adsorption with time was estimated to determine the system equilibrium (see FIG. 7A). It attains equilibrium in 2 h with minimal variations thereafter. The solvent-polymer interaction dictates adsorption and potentially causes the observed variation (see below). The Redlich-Peterson model adequately describes the adsorption of chlorogenic acid on P2 (FIG. 7B), indicating monolayer followed by multilayer adsorption consistent with the BET isotherm. The exponent of 0.6 (see Table 8 for regression coefficients) indicates a relatively heterogeneous surface.

TABLE 8
Fitted adsorption parameters of polymer P2
using Redlich-Peterson models at 298 K.

| Parameters | α (L/mg) | Kᵣ (L/g) | β | R² |
|---|---|---|---|---|
| Redlich-Peterson | 188.40 | 1978.76 | 0.60 | 0.92 |

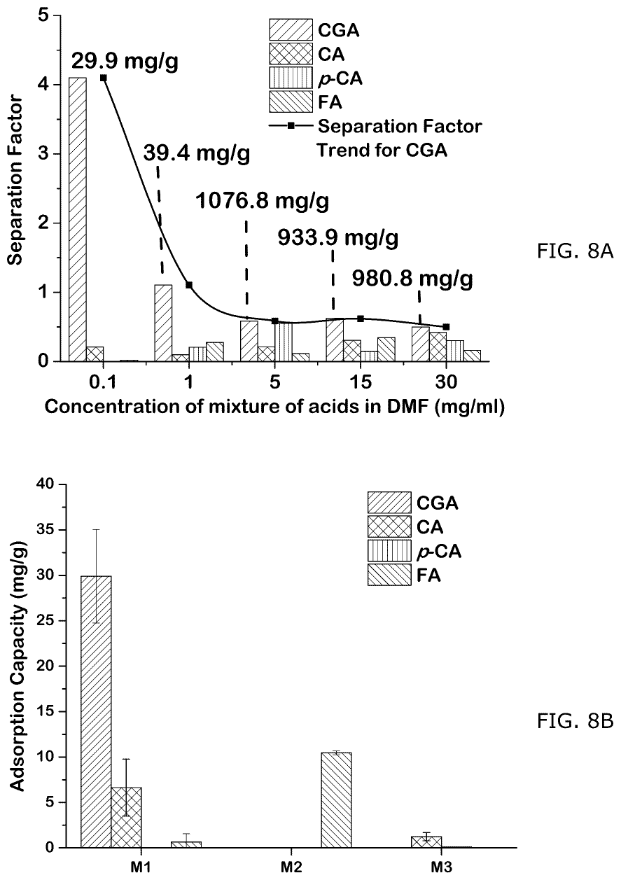

Additionally, P2's efficacy for a concentrated mixture of target acids was measured. The polymer is highly selective to chlorogenic acid at concentrations as low as 0.1 pg/mL mixture of acids in DMF (see below). On the contrary, P2's selectivity decreases steeply between 0.1 to 5 mg/mL and plateaus at higher concentrations (FIG. 8A). Without wishing to be bound by any particular theory, it is believed that non-selective pores on P2, due to its heterogeneous surface, cause this behavior. The selective pores of P2 get occupied at lower analyte concentrations leaving only non-selective pores. These pores behave like a non-imprinted polymer, showing no preference for any analyte. P2 is still highly efficient at low concentrations extracted from FW (˜0.01 to 20 μg/mL). A stagewise adsorption process with an extraction unit (see process flow diagram in FIG. 1) using P2 can provide high purity of target compounds at each stage. According to various embodiments, controlled/living radical polymerization techniques, such as reversible addition-fragmentation chain transfer, iniferter, and atom transfer radical polymerization, provide efficient alternatives to the traditionally time-intensive free radical polymerization.

Further, P2's selectivity in mixtures in stagewise adsorption was examined (FIG. 8B). Three standard mixtures (M1, M2, and M3) of different components were prepared. When all four phenolics are present (M1), P2 displays the highest selectivity toward chlorogenic acid. It is selective to ferulic acid when only three phenolics except chlorogenic acid are present (M2). When only p-coumaric and caffeic acid are present, it is selective to the latter. The P2's selectivity in mixtures with the highest adsorbing acid removed each time is:

chlorogenic acid>ferulic acid>caffeic acid>p-coumaric acid

P2's performance depends on the polarity and HB interactions with the adsorbate. As discussed above, chlorogenic and p-coumaric acid have the most and least HB sites, respectively. Due to this and chlorogenic acid being the imprinted molecule, it is favored by P2. Similarly, P2 has the lowest selectivity towards p-coumaric acid. Structurally, ferulic acid has an HB acceptor group (O—CH₃), while caffeic acid has an HB donor group (O—H). The HB donor active site and itaconic acid interact more strongly with the ferulic acid than the caffeic acid. Hence, P2 can purify the target compounds consecutively due to its responsiveness to different functionalities.

### P2's Performance and Stability in DMF and Common Extraction Solvents

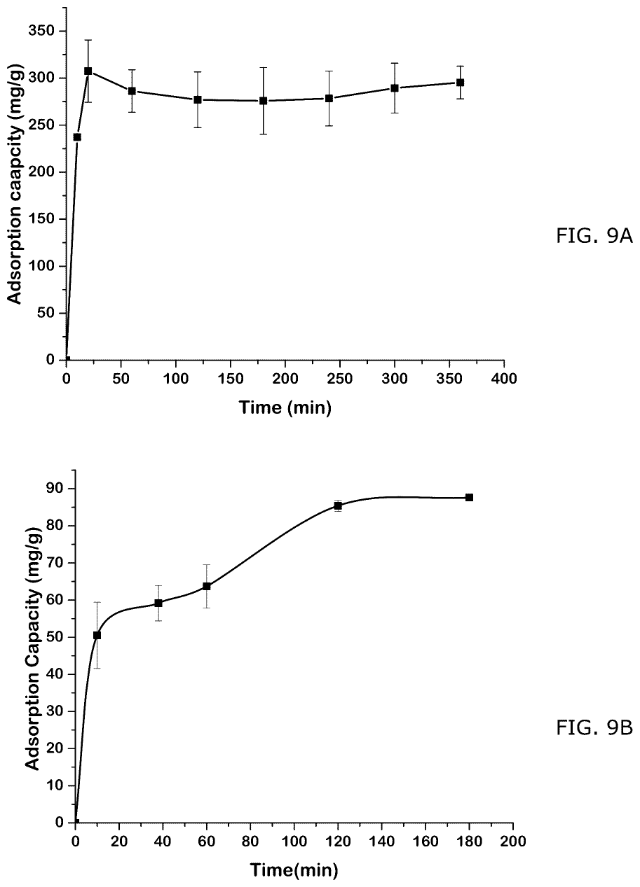

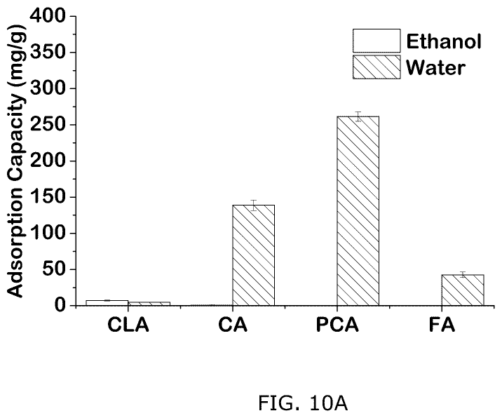

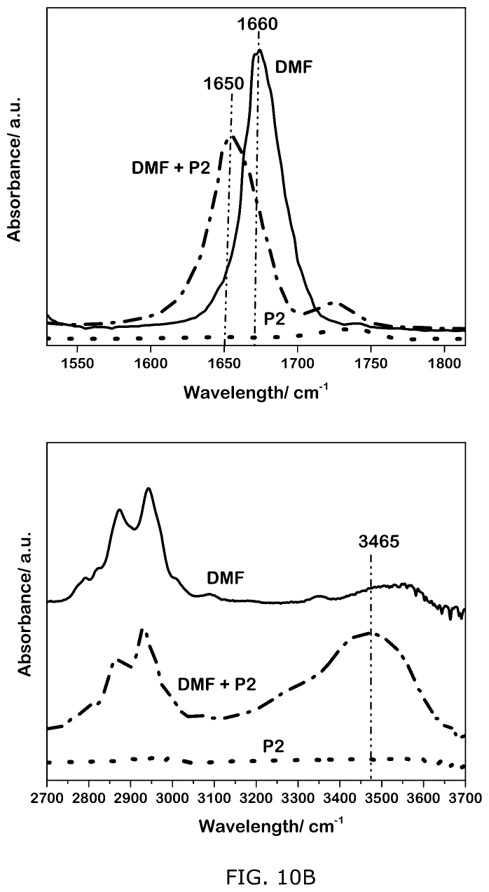

Ethanol and water are common solvents used for extracting bioactive compounds from FW, plants, and biomass. DMF is a polar aprotic solvent with good HB acceptor character that efficiently extracts target compounds from the aforestated feedstocks. Thus, P2's performance and stability was tested in these three solvents to evaluate its scope for diverse applications. FIGS. 9A and 9B show time-dependent single component adsorption capacity of P2 for 1 mg/ml chlorogenic acid at 298 K in water and ethanol respectively. Water provides the highest single-component adsorption capacity (FIG. 9A 307.36±33.02 mg/g) of chlorogenic acid (1 mg/mL) on P2, followed by DMF (Figure not shown; 148. 38±6.58 mg/g), and ethanol (FIG. 9B; 85.39±1.46 mg/g). P2 displays nominal separation efficiency for all acids during multicomponent adsorption in water with the highest selectivity towards p-coumaric acid (separation factor=1.4) (see Table 9 for separation factors in water and ethanol). It selectively separates chlorogenic acid from a multicomponent mixture in ethanol (separation factor=8.72) (FIG. 10A). Therefore, P2 has superior performance in ethanol, followed by DMF, and lastly, water.

TABLE 9
Separation factor of target extractives for polymer
P2 for a mixture containing an equal concentration
of chlorogenic, caffeic, p-coumaric, and ferulic acid
at 0.1 mg/ml in ethanol and water at 298 K and 2 h.

| | Separation | | Separation | |
|---|---|---|---|---|
| | factor for | Separation | factor for | Separation |
| | chlorogenic | factor for | p-coumaric | factor for |
| Solvent | acid | caffeic acid | acid | ferulic acid |
| Ethanol | 8.72 | 0.08 | 0.01 | 0.02 |
| Water | 0.01 | 0.45 | 1.40 | 0.11 |

The solvent effect on the adsorbent separation efficiency is well-studied in the literature. The hydrophobic/hydrophilic interaction of the extraction solvent with the adsorbent affects its performance. When adsorption is carried out in the solvent used for imprinting, the adsorbent's binding efficiency is enhanced due to a stable cavity structure. Conversely, the polymer solvation in other solvents can lead to unstable conformations and changes in the cavity structure. FT-IR was used to study the interactions of the selected solvents with P2 (not shown). Ethanol and water have strong HB character due to hydroxyls, which limits the identification of HB with IR. For P2 in DMF (FIG. 10B), it was observed that the C═O band of DMF at 1660 cm⁻¹ shifts to lower wavenumber (approximately 10 cm⁻¹), and a new O—H stretching band at 3465 cm⁻¹ appears. A similar peak shift (approximately 7 cm⁻¹) of the C═O band and the appearance of the 0-H band (3260 cm⁻¹) is seen when mixtures of DMF and chlorogenic acid are analyzed (not shown). This indicates that the C═O band of DMF accepts HB from P2 (—COON, —OH) or chlorogenic acid, which can influence P2's cavity structure and performance. Alternatively, polymer swelling can alter the cavity structure leading to low selectivity. The polymer swelling (see above) is relatively low due to the high crosslinker amount that governs the pore size and selectivity. The currently used 1:3 monomer-to-crosslinker ratio is widely accepted for non-covalent imprinting. Without wishing to be bound by any particular theory, it is believed that a further increase in the crosslinker amount can lead to reduced pore size and selectivity. The inherent HB and high swelling ratio of P2 in DMF caused the observed fluctuations over time (FIG. 7A), leading to large error bars in adsorption capacity. The weak interaction of P2 with ethanol compared to DMF, evidenced by its lower swelling ratio in ethanol is likely responsible for the observed higher selectivity due to minimal changes in the cavity structure. Further, both P2 and target compounds exhibit weak interactions with water. As a result, these compounds tend to adsorb on P2 instead, making separation less selective in water.

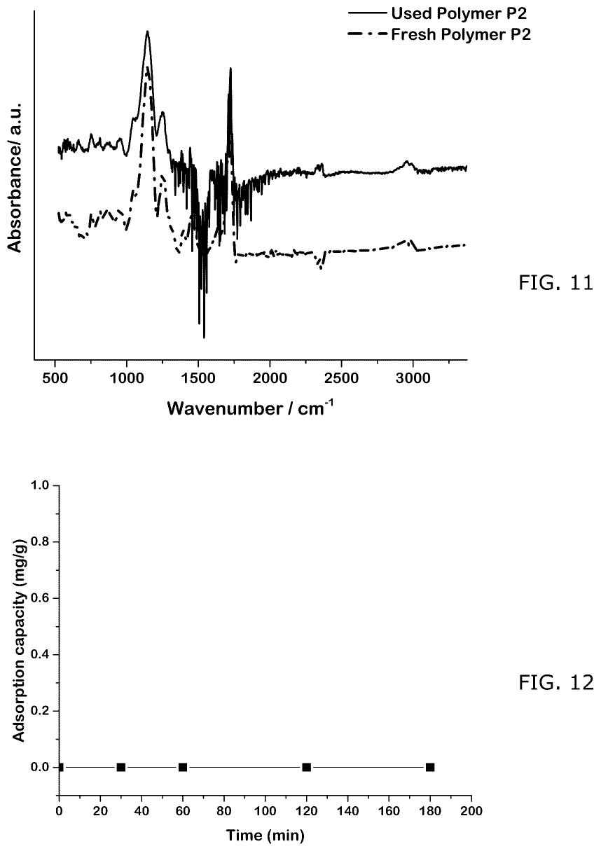

The observed approximately 15% variability in adsorption capacity in DMF was further investigated (see FIG. 7A for time-dependent adsorption). P2 is chemically stable in DMF, indicating no degradation and loss of chemical structure (see FIG. 11 for the IR spectrum of used polymer P2). In addition, DMF does not participate in competitive adsorption with the target analytes (FIG. 12). Thus, it is believed that the inherent HB between P2 and DMF and the high swelling ratio cause the observed fluctuations over time.

### Chemical Stability of P2 in DMF

Structural changes leading to loss of recognition sites can occur if DMF reacts or degrades P2. FIG. 11 shows the IR spectrum of a fresh and spent (used) polymer (P2 after one cycle of adsorption at 0.1 mg/ml chlorogenic acid in DMF). The IR spectrum shows that all the characteristic peaks of the fresh polymer overlap with those of the spent polymer. The polymer representative peaks of itaconic acid and EDGMA are at 1640 cm⁻¹ (C═C stretching band), 1721 cm⁻¹ (C═O stretching band), and 1150 cm⁻¹ (C—O—C). This signifies that P2 is stable in DMF with no degradation or loss of chemical structure.

### Adsorption of DMF on P2

The adsorption of DMF on polymer P2 was further investigated. A stock solution of 0.65 mg/ml of DMF in ethanol was made. 2 mg of polymer was added to a vial containing 2 ml of stock solution. The mixture was stirred at 298 K for 30, 60, 120, and 180 min. All experiments were run in triplicates and analyzed on HPLC. 0.45 μm syringe filters were used for sampling from each vial.

FIG. 12 shows no adsorption of DMF on P2 for 3 h. This signifies that P2 is not adsorbing DMF and is selective towards chlorogenic acid in the mixture. If DMF participates in competitive adsorption with chlorogenic acid, P2 selectivity towards the target analyte would be significantly compromised, rendering itself nugatory.

### Comparative Example 1

### Comparison of Non-Imprinted and Imprinted Polymers

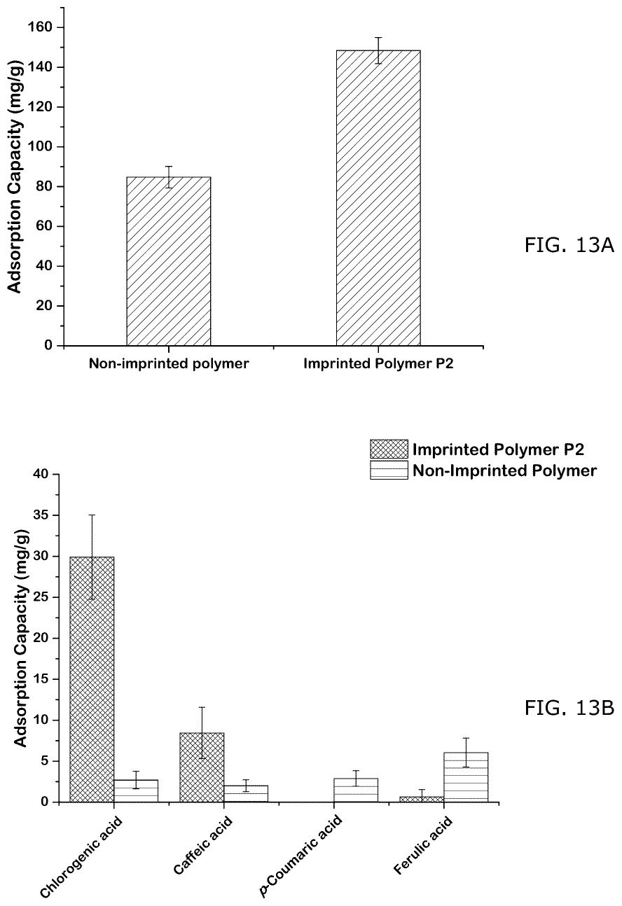

The performance of imprinted (MIP) and non-imprinted polymers for single and multicomponent adsorption was tested and compared. The imprinted polymer provides ˜approximately 1.7 times higher adsorption capacity for chlorogenic acid than the non-imprinted in single component adsorption experiments (FIG. 13A). It provides the highest reported impact factor (see Eq. 4) of 11.1 in multicomponent adsorption. The surface morphology of non-imprinted polymer and P2 (FIGS. 6B and 6C) demonstrates the successful formation of porous structures. The non-imprinted polymer has a higher surface area (approximately 1.5 times that of P2) but a similar pore size to P2 (Table 6). The non-imprinted polymer is non-selective even with a higher surface area, displaying nearly equal adsorption for all analytes in the mixture (FIG. 13B). This indicates the presence of fewer but selective cavities on P2 due to effective imprinting resulting in its superior performance.

Impact factor=q_MIP/q_NIP (4)
where q_MIP and q_NIP are the molecule's adsorption capacity by the imprinted polymer and non-imprinted polymer, respectively.

Considering the complex chromatograms of coffee waste and potato peel extracts (not shown), we utilized standards to determine the retention times of target acids. Extracted ion chromatograms (not shown) were then employed to detect phenolics in the extracted solutions.

### Example 2

### Reusability

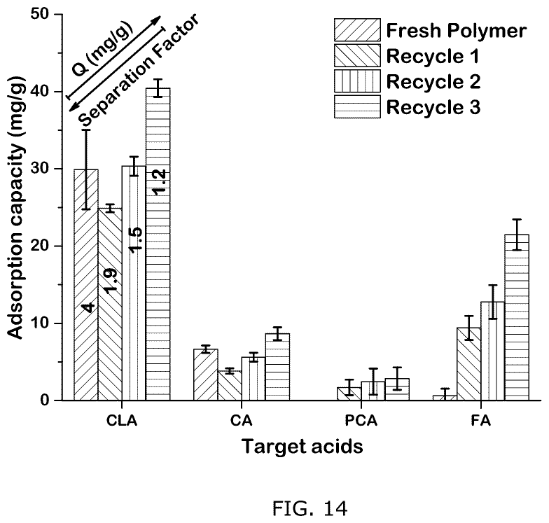

P2 of Example 1 was tested for its reusability. P2 was collected after batch adsorption up to three times, washed with methanol, and dried under vacuum. Its adsorption capacity increased with every recycle, but the selectivity decreased (FIG. 14). It still provides nominal selectivity for up to three recycles. The separation factor reduces by ˜approximately 50% after the first recycling and ˜approximately 20% in the subsequent cycles. As discussed above, P2 has limited stability in DMF and, thus, loses selective pores. The polymer reuse could also rupture the selective pores due to the lack of P2's stability in different solvents.

### Example 3

### Application to Real FW

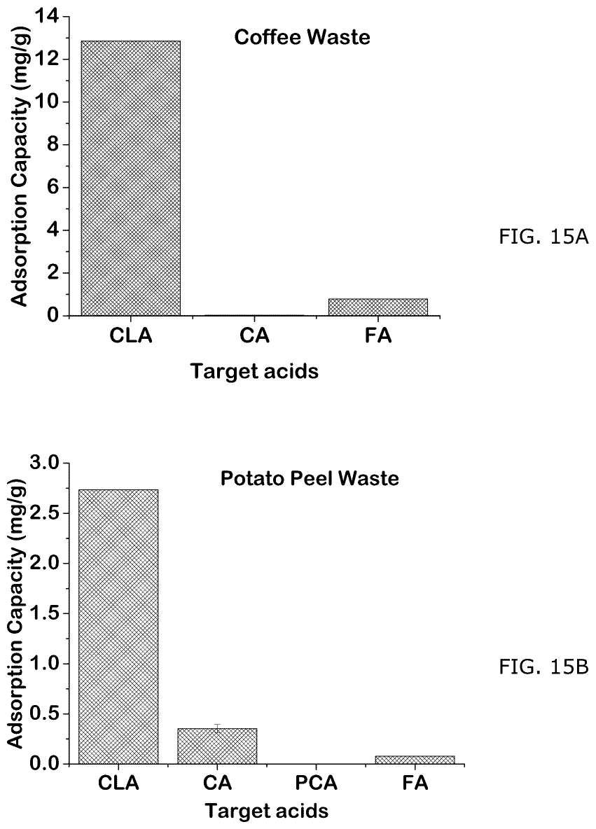

Potato peel waste (PPW) and spent coffee bean waste (CW) extracts in DMF were prepared (see methods) to examine P2's performance. All four target compounds were detected in PPW and only three (except p-coumaric acid) were detected in CW (see the concentration of target acids in Table 10) using UPLC-MS (see Methods). P2 selectively purifies 86% and 92% chlorogenic acid in one cycle from PPW and coffee waste due to higher concentrations of chlorogenic acid in the selected feedstocks (FIGS. 15A and 15B). P2 can provide an even higher purity of chlorogenic acid (>99%) in subsequent cycles.

TABLE 10
Target acids concentration extracted from potato peel
and spent coffee bean waste using DMF at 298 K for
2 h at 60° C. and 1:20 solid-to-liquid ratio.

| | Chlorogenic | Caffeic | p-Coumaric | Ferulic |
|---|---|---|---|---|
| | acid | acid | acid | acid |
| Food waste | (μg/ml) | (μg/ml) | (μg/ml) | (μg/ml) |
| Potato peel waste | 2.735 | 1.381 | 0.056 | 0.078 |
| Spent coffee bean | 12.853 | 0.237 | — | 7.891 |
| waste | | | | |

### Lab Scale Economic and Carbon Footprint Analysis

Various grades of chlorogenic acid are produced commercially and priced based on purity. The 45% and 50% pure chlorogenic acid is valued at 0.16 USD and 0.28 USD per gram, whereas pure chlorogenic acid per USP reference standard and 95% pure chlorogenic acid cost 7,220 USD/g and 122 USD/g.

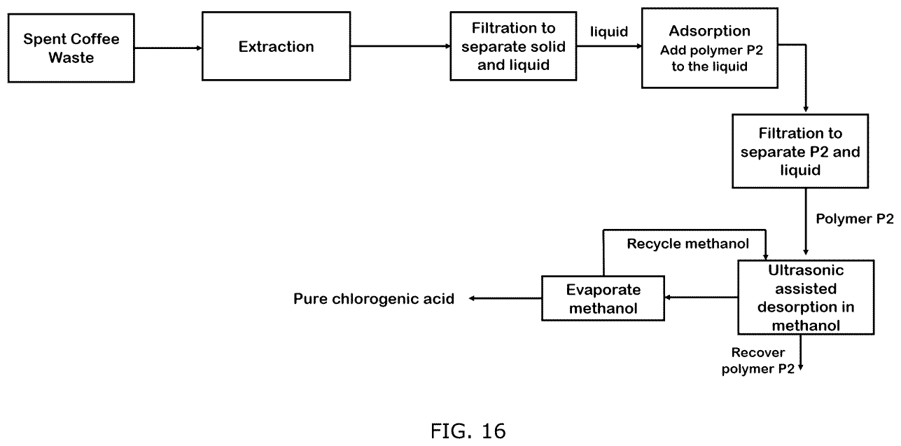

The cost for producing pure chlorogenic acid from spent coffee beans using P2 with a simple lab-scale economic analysis as (CGA is chlorogenic acid) was estimated (see FIG. 16 for the process flow diagram):

Cost of purifying CGA=cost of (CGA extraction+P2 synthesis+Separation)

The process costs are calculated as follows:

Total Cost=cost of (raw materials+extraction+separation)

The cost of extracting chlorogenic acid is estimated at 0.13 USD/g. The costs of the raw materials are presented in Table 11. One lab-scale P2 synthesis reaction gives approximately 4 g polymer. The reaction details are in the materials and methods section of the text. The total cost of synthesizing 1 g polymer is 5.71 USD. The energy costs associated with conducting lab-scale separation at 298 K for 2 h was considered, but did not account for the energy required for solvent evaporation or the cost of solvent make-up through recycling. With the separation efficiency of 92% pure chlorogenic acid in one cycle, the separation cost is 5.14 USD/g chlorogenic acid.

TABLE 11
The raw material cost for polymer synthesis.

| | | | | Price | Final |
|---|---|---|---|---|---|
| | | | | (USD/ | Cost |
| Material | Compound | Amount | | unit) | (USD) |
| Monomer | Itaconic acid | 0.47 | g | 0.056/g | 0.0264 |
| Solvent | THF | 5 | mL | 0.096/mL | 0.4825 |
| Crosslinker | Ethylene glycol | 2.07 | mL | 0.018/mL | 0.0376 |
| | dimethacrylate | | | | |
| Initiator | Azobisisobutyronitrile | 0.03 | g | 2.62/g | 0.0786 |
| Template | Chlorogenic acid | 0.32 | g | 69.40/g | 22.2080 |

Burniol-Figols et al. (Biochem Eng J. 116 (2016) 54-64) obtained an extraction efficiency of 54.6±0.7 mg for chlorogenic acid/ g from spent coffee beans using 60% (v/v) ethanol at solid-to-liquid ratio of 1:10 and 70° C. for 40 min at 0.13 USD/g. Kao Corporation produces a mixture of nine monocaffeoylquinic, feruloylquinic, and dicaffeoylquinic acids giving 11% pure chlorogenic acid (1/9×100) whereas the presently disclosed methodology produces 92% pure chlorogenic acid. The U.S. Pat. No. 8,309,150 of Kao Corporation uses a liquid passing factor of 6 (v/v) for extract solution and adsorbent volume resulting in a chlorogenic acid adsorption capacity of 2.1 mg/g whereas the presently disclosed process provides chlorogenic acid adsorption capacity of 12.8 mg/g. Kao Corporation employed a space velocity of 5.2 mL/g/h amounting to 40 min for producing 6.0 mg pure chlorogenic acid. In contrast, the presently disclosed process purifies 50.23 mg of chlorogenic acid in 30 min. An emission factor of 0.29 kg/kWh is taken for ultrasonic adsorption and chromatographic separation. The carbon footprint of the three steps for both methods is given in Table 13.

One lab-scale P2 synthesis reaction and separation cycle gives ˜4 g polymer and 92% pure chlorogenic acid. The present analysis estimates the cost of obtaining 1 g of pure chlorogenic acid at 11.00 USD/g (Table 12). Hence, the presently disclosed process provides at least 11 times more economic value than the commercially available product at 95% purity grade.

TABLE 12
Cost distribution of lab-scale extraction and purification
of chlorogenic acid from spent coffee bean waste.

| Expense (per gram) | Cost (USD) |
|---|---|
| Polymer synthesis | 5.71 |
| Chlorogenic acid extraction | 0.13 |
| Chlorogenic acid separation | 5.14 |
| Total cost (USD /g) | 11.00 |

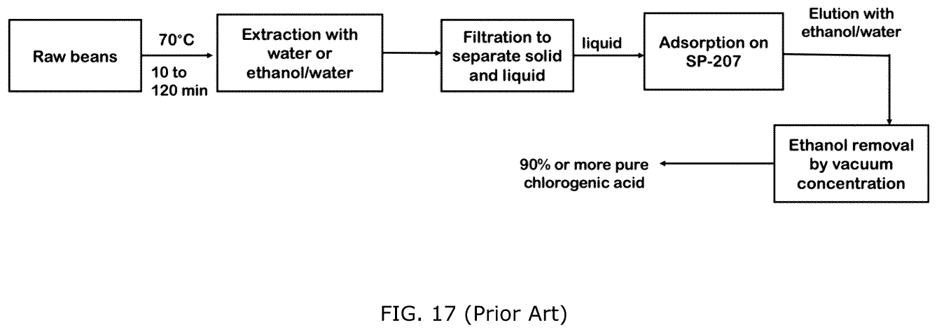

Further, the carbon footprint of the presently disclosed process (see FIG. 16) was evaluated and contrasted it with Kao Corporation's (see FIG. 17). The presently disclosed process offers three distinctions: a) choice of feedstock, b) adsorbent, and c) recovery method. In contrast, the Kao corporation utilizes raw coffee beans with nearly the same chlorogenic acid amount found in the waste and a higher carbon footprint. (Ind Crops Prod. 50 (2013) 423-429; Food Chem. 64 (1999) 547-554) An assumption that both methods' adsorbent synthesis carbon emissions are roughly equivalent was made and the emissions from the monomer production were compared. SP-207 adsorbent consists of a brominated styrene-divinylbenzene matrix synthesized from benzene (monomer). The presently disclosed process incorporates ultrasonic desorption and vacuum concentration as the recovery process, whereas Kao Corporation's recovery process involves column washing followed by vacuum concentration. Consequently, the presently disclosed process reduces carbon emissions by at least approximately 95% (see Table 13 for detailed calculations) by utilizing waste-derived feedstock and monomer and fosters a circular economy to produce high-value chemicals.

TABLE 13
Carbon footprint of the industrial and proposed technology
for extraction and purification of chlorogenic acid.

| | Kao Corporation Technology | | Proposed Technology | |
|---|---|---|---|---|
| | Footprint | | Carbon Footprint | |
| Metric | (kg CO₂e) | | (kg CO₂e) | |
| Feedstock | Raw coffee | 14-16/kg green | Spent coffee | 0 |
| | beans | coffee powder | beans | |
| Monomer | SP-207 | 1.76/kg | P2 | 0/kg |
| Production | | benzene | | itaconic |
| | | | | acid |
| Recovery | Elution on | 0.06/kWh | Batch | 0.03/kWh |
| | column | | ultrasonic | |
| | | | desorption | |
| Total | Per g | 3.32-3.36 | Per g | 0.07 |
| | chlorogenic | | chlorogenic | |
| | acid | | acid | |

TABLE 14
Phenolic acids mixtures produced by different companies.

| Company | Product name | Assay |
|---|---|---|
| Applied Food Sciences (Applied Food Sciences, | Green Coffee | 50% chlorogenic |
| Green Coffee Extract, Applied Food Sciences. | Extract | acids, caffeine <4% |
| (2022);https://appliedfoods.com/ingredients/gca/) | | |
| NutraGreen (NutraGreen, Green Coffee bean | Green Coffee | Chlorogenic acids |
| Extract Chlorogenic Acids, NutraGreen. (2022); | bean extract | 60%, 50%, 25% |
| https://www.nutragreenbio.com/product/green- | chlorogenic | |
| coffee-bean-extract-chlorogenic-acids) | acids | |
| Euromed (Euromed, Artichoke Extracts, 2022; | Artichoke Dry | >15% |
| https://www.euromedgroup.com/product- | extract | caffeoylquinic |
| details/artichoke-leaf-cynara-scolymus-l/) | | acids; <4% |
| | | caffeoylquinic acids |
| Cymbio Pharma (Cymbio Pharma, Herbal Extracts, | Green Coffee | Chlorogenic acids |
| Cymbio Pharma. (2022); | Bean Extract | in grades of 45%, |
| https://www.cymbio.co.in/products/herbal- | | 50%, 60% |
| extracts/) | | |
| Flavour Trove (Flavour Trove, Human Health Care, | Green Coffee | 40-60% |
| Flavour Trove. (2022); | Extract (Coffee | chlorogenic acid |
| https://www.flavourtrove.com/(accessed Aug. | Arabica) | |
| 17, 2023)) | | |

### Conclusions

Extracting high commercial-value bioactive compounds, such as phenolic acids, from FW and non-food biomass is an attractive method to repurpose FW. However, the separation and downstream purification of the extracted compounds is challenging. As disclosed hereinabove, molecular imprinted polymers were synthesized to selectively separate the target phenolic acid compounds. HSPiP was used to screen 13 porogenic solvents and 28 monomers and THF was identified as the best solvent for polymer synthesis. Experimental investigations revealed that itaconic acid stands out as an efficient and green alternative, providing the highest separation factor for chlorogenic acid compared to previous studies. The selectivity of the optimized polymer (P2) was found to be chlorogenic acid>ferulic acid>caffeic acid>p-coumaric acid. Further, P2 is selective up to 1 mg/mL equal concentration acids mixture in DMF, which is much higher than in FW, offering a broader range of operations for the polymer and extending its utility beyond traditional analytical applications. Three distinct extraction solvents were evaluated, with ethanol providing the highest separation factor for chlorogenic acid, followed by DMF, and then water. IR was used to understand how P2 interacts with different solvents. The high swelling ratio in DMF indicates the possible collapse of selective pores. Its application to a real FW reveals successful extraction of up to 92% and 86% pure chlorogenic acid from coffee beans and potato peel waste, respectively.). The process of the present invention provides superior economic value and significantly less carbon emissions compared to conventional processes, such as disclosed in U.S. Pat. No. 8,309,150. The application of waste-derived monomer and feedstock assists in offsetting carbon emissions and drives circularity.

What is claimed:

1. A process for separating phenolic acids, comprising:
a) contacting a feed containing at least two different phenolic acids (PAs) with an extraction solvent to extract the at least two different phenolic acids in a first PA containing liquid;
b) contacting the first PA containing liquid with a solid molecular imprinted polymer (MIP), such that the MIP captures a target phenolic acid from the at least two different phenolic acids, to thereby form a first phenolic acid bound MIP dispersed in a second PA containing liquid, wherein the second PA containing liquid comprises at least one phenolic acid and none or a substantially lesser amount of the target phenolic acid originally present in the first PA containing liquid;
c) separating the first phenolic acid bound MIP from the second PA containing liquid;
d) separating the target phenolic acid from the first phenolic acid bound MIP to obtain a recovered MIP, wherein the recovered MIP is substantially free of the target phenolic acid;
e) optionally repeating the steps b) to d) to extract remaining phenolic acid(s) of the at least two phenolic acids.

2. The process according to claim 1, wherein the at least two phenolic acids differ from each other in at least one of functional group, aromatic ring substitution, polarity, and hydrogen bonding.

3. The process according to claim 1, wherein the target phenolic acid is chlorogenic acid.

4. The process according to claim 3, wherein the at least two different phenolic acids further comprises one or more of caffeic acid, p-coumaric acid, and ferulic acid.

5. The process according to claim 1, wherein the MIP in a second sequence of steps b) to d) is the recovered MIP.

6. The process according to claim 1, wherein the target phenolic acid has a separation factor of at least 1, wherein the separation factor is calculated as follows:
$$Seperation factor = \frac{Adsorption capacity of target phenolic acid}{\begin{matrix}Sum of adsorption capacity of all other phenolic \\ acids of the at least two phenolic acids\end{matrix}} ;$$

7. The process according to claim 1, wherein the MIP has a BET surface area in the range of 80 to 250 m²/g and a BET pore size in the range of 5 to 11 nm.

8. The process according to claim 1, wherein the feed comprises one or more of raw, roasted, or spent coffee beans; potato peels; grapes; honeysuckle; apple; tomato; eggplant; carrot; and leaves from artichoke, E. ulmodies, tea, and tobacco.

9. The process according to claim 1, wherein the target phenolic acid is extracted at a purity of at least 80%.

10. The process according to claim 1, wherein the step of separating the target phenolic acid from the phenolic acid bound MIP comprises using ultrasonic assisted desorption in methanol, ethanol, 2-propanol, and/or tetrahydrofuran.

11. The process according to claim 1 further comprising preparing the MIP, comprising the steps of:
(i) providing a polymerizable mixture comprising a pre-polymerization complex of at least one functional monomer and a target phenolic acid in at least one porogenic solvent;
(ii) polymerizing the polymerizable mixture in the presence of a cross-linker, and the porogenic solvent to generate the MIP, wherein the target phenolic acid is non-covalently bound to the polymer;
(iii) washing the MIP with an extraction solvent to remove the target phenolic acid and to thereby form a MIP comprising molecular sized cavities adapted to selectively capture and bind the target phenolic acid.

12. The process according to claim 11, wherein the step of providing a polymerizable mixture comprises selecting the at least one functional monomer and at least one porogenic solvent based on their respective molecular interactions with the target phenolic acid.

13. The process according to claim 12, wherein the target phenolic acid has a solubility in the at least one porogenic solvent in a mole fraction range of 0.001 to 0.99, based on the total moles of the target phenolic acid and the porogenic solvent.

14. The process according to claim 12, wherein the at least one functional monomer has a solubility in the at least one porogenic solvent in a mole fraction range of 0.01 to 0.99.

15. The process according to claim 12, wherein the at least one porogenic solvent has a dielectric constant in a range of 5 to 50.

16. The process according to claim 12, wherein the at least one functional monomer has the following Hansen Solubility parameters:
(i) a dispersion δD in the range of 15 to 21;
(ii) a polarity δP in the range of 5 to 15;
(iii) a hydrogen bond character δH in the range of 7 to 21; and
(iv) a Hansen Solubility Parameters in Practice (HSPiP) distance from the target phenolic acid in the range of 0 to 10.

17. The process according to claim 11, wherein the at least one functional monomer is selected from the group consisting of acrylamide, 4-vinyl pyridine, 2,6-diaminopyridine, itaconic acid, o-phenylenediamine, o-aminophenol, 2-hydroxyethyl methacrylate, p-aminostyrene, o-phthalic dialdehyde, acrylic acid, methacrylamide, N,N′-methylene bisacrylamide, methacrylic acid, N,N-dimethylacrylamide, allyl mercaptan, p-divinylbenzene, acrolein, 2-vinyl pyridine, N-vinyl-2-pyrrolidinone, acrylonitrile, methyl methacrylate, styrene, N,N-dimethylaminoethyl methacrylate, 4-ethyl styrene, (diethylamino)ethyl methacrylate, m-divinylbenzene, 3-aminopropyltriethoxysilane, tartaric acid, lactic acid, and combinations thereof.

18. The process according to claim 11, wherein the at least one porogenic solvent comprises hexane, benzene, toluene, chloroform, tetrahydrofuran, dichloroethane, dichloromethane, 2-methoxyethanol, ethanol, methanol, N,N-dimethylformamide, acetonitrile, dimethyl sulfoxide, or mixtures thereof.

19. The process according to claim 11, wherein the at least one porogenic solvent comprises tetrahydrofuran, and the at least one functional monomer comprises acrylamide, o-aminophenol, itaconic acid, o-phenylenediamine, 2-hydroxyethyl methacrylate, or combinations thereof.

20. The process according to claim 11, wherein the target phenolic acid comprises chlorogenic acid, caffeic acid, p-coumaric acid, or ferulic acid, wherein the at least one functional monomer comprises itaconic acid, wherein the radical initiator comprises 2,2-azobisisobutyronitrile, and wherein the crosslinker comprises ethylene glycol dimethacrylate and/or 1,3-diisopropylbenzene.

21. The process according to claim 11, wherein the target phenolic acid and the functional monomer are present at a ratio in the range of 1:2 to 1:6.

22. The process according to claim 11, wherein the functional monomer and the crosslinker are present at a ratio in the range of 1:1 to 1:5.
